(19) United States
(12) Patent Application Publication
(10) Pub. No.: US 2020/0000679 A1
(43) Pub. Date: Jan. 2, 2020

(54) Cardiopulmonary Resuscitation Using Networked Devices

(71) Applicant: Ximio Health Inc., Wilmington, DE (US)

(72) Inventor: Beno Oppenheimer, New York, NY (US)

(21) Appl. No.: 16/021,906

(22) Filed: Jun. 28, 2018

(51) Int. Cl. A61H 31/00; A61N 1/39

(52) U.S. Cl. CPC A61H 31/005; A61H 2201/5069; A61N 1/39044; A61N 1/3987; A61N 1/3993; A61H 2201/10; A61H 2201/1253; A61H 2201/1621; A61H 2201/5012; A61H 2205/084; A61H 2201/169; A61H 2201/5084; A61H 2230/255; A61H 2201/5089; A61H 2230/305; A61H 2201/5043; A61H 31/007

(57) ABSTRACT

A a system includes a first computing device and a chest compression device. The chest compression device is configured to communicate with the first computing device. The chest compression device can include a defibrillator. The first computing device is configured to obtain information regarding a patient being treated for cardiopulmonary arrest and to send commands to the chest compression device. The commands include a defibrillator activation command to activate the defibrillator.

### TECHNICAL FIELD

The present disclosure relates to cardiopulmonary resuscitation using networked devices.

### BACKGROUND

Cardiac arrest, also known as cardiopulmonary arrest or circulatory arrest, is a sudden stop in effective blood circulation due to the failure of the heart to contract effectively or at all. Cardiac arrest is often treated via attempts at resuscitation. However, due to the acuity of these situations, the patient's outcome depends greatly on the good decision making and the timing of his caretakers.

To improve the likelihood of success, caretakers often follow guidelines for decision making and timing of interventions. For example, the American Heart Association has created intervention guidelines called the Advanced Cardiac Life Support (ACLS) protocols. Many hospitals mandate that providers be trained in these life-saving protocols.

### SUMMARY

In general, in an aspect, a system includes a first computing device and a chest compression device. The chest compression device is configured to communicate with the first computing device. The chest compression device includes a defibrillator. The first computing device is configured to obtain information regarding a patient being treated for cardiopulmonary arrest and to send commands to the chest compression device. The commands include a defibrillator activation command to activate the defibrillator.

Implementations of this aspect can include one or more of the follow features.

In some implementations, the chest compression device includes: a compression plate; a compression bladder; and at least one adjustable strap coupled to the compression plate and for securing the chest compression device to the patient. The defibrillator may include: a first defibrillator pad attached to the compression bladder; and a second defibrillator pad attached to the at least one adjustable strap. The compression bladder may be configured to be removably secured to the compression plate. The compression bladder may be secured to the compression plate through a hook and loop fastener. The chest compression device may include a gel pack. An internal space of the compression bladder may include a gel or liquid. The system may further include a compressor couplable to the compression bladder, in which, during operation of the system, the compressor is operable to inflate and deflate the compression bladder. The chest compression device may include a power source coupled to a first defibrillator pad. The chest compression device may be configured to cause the power source to apply a voltage potential to the first defibrillator plate upon receiving the defibrillator activation command from the first computing device.

In some implementations, the chest compression device includes an accelerometer configured to measure an acceleration of the chest compression device during operation of the chest compression device, in which the first computing device is configured to obtain, from the accelerometer, an acceleration signal indicative of the acceleration and to derive a compression depth associated with treating the patient based on the acceleration signal. The accelerometer may be embedded within the compression plate. The chest compression device may be operable to automatically apply pressure to the patient periodically.

In some implementations, the system further includes a capnometer operable to sense a concentration of carbon dioxide exhaled by the patient, in which the capnometer is couplable to the first computing device.

In some implementations, the system further includes an oxygen sensor operable to sense an amount of oxygen perfusion of the patient, in which the oxygen sensor is couplable to the first computing device.

In some implementations, the system further includes a tissue perfusion sensor operable to sense an amount of tissue perfusion of the patient, in which the tissue perfusion sensor is couplable to the first computing device.

In some implementations, the system further includes a brain oxygenation sensor operable to sense an amount of brain oxygenation of the patient, in which the brain oxygenation sensor is couplable to the first computing device.

In some implementations, the system further includes a blood pressure sensor operable to sense a blood pressure of the patient, in which the blood pressure sensor is couplable to the first computing device.

In some implementations, the chest compression device includes an angular rate sensor.

In some implementations, the chest compression device includes one or more light emitting elements.

In some implementations, the the chest compression device includes: an upper compression plate; a lower compression plate; and a mechanical piston between the upper compression plate and the ower compression plate and configured to increase a distance between the upper compression plate and the lower compression plate during operation of the chest compression device.

In some implementations, the system further includes: a strap coupled to the chest compression device; and a strap tensioner for receiving a first end of the strap and a second end of the strap such that the strap is arranged in a loop, in which, during operation of the the chest compression device, the strap tensioner is operable to increase or decrease a length of the loop.

In some implementations, the chest compression device can wirelessly communicate with the first computing device.

In some implementations, the chest compression device can be configured to allow a user to manually perform chest compressions on the patient. The first computing device or the chest compression device can be configured to display a prompt or information regarding the patient to the user.

In some implementations, the chest compression device can include a compression plate, a mechanical piston, and an adjustable strap coupled to the compression device. The strap can secure the chest compression device to the patient. The mechanical piston can be configured to compress, via the compression plate, the chest of the patient. The mechanical piston can be positioned directly on the compression plate, or can be connected to the compression plate via a connector.

In some implementations, the chest compression device can include a compression plate, a compression bladder, and an adjustable strap coupled to the compression plate. The strap can secure the chest compression device to the patient. The compression bladder can be coupled to a compressor configured to inflate and deflate the compression bladder. The compressor can inflate and deflate the compression bladder using any suitable gas (e.g., air or oxygen).

In some implementations, the defibrillator can include a first defibrillator pad attached to the compression bladder and a second defibrillator pad attached to the adjustable strap. The compression bladder can be configured to be removably secured to the compression plate (e.g., through a hook and loop fastener).

In some implementations, the defibrillator includes a power source coupled to a first defibrillator pad. The chest compression device can be configured to cause the power source to charge the first defibrillator pad upon receiving the defibrillator activation command from the first computing device.

In some implementations, the chest compression device can include a pressure sensor configured to measure a pressure applied by the chest compression device to the patient during operation of the chest compression device. The first computing device can be configured to obtain, from the chest compression device, the pressure measured by the pressure sensor and a compression depth associated with treating the patient. The pressure sensor can be embedded within the compression plate. The chest compression device can apply the pressure to the patient in a synchronized or oscillating manner.

In some implementations, the chest compression device can include an electrocardiograph. The first computing device can be configured to obtain information regarding the patient being treated for cardiopulmonary arrest from the electrocardiograph.

In some implementations, the chest compression device can include a sensor and an electronic control module in communication with the sensor. The electronic control module can be configured to obtain a measurement signal from the sensor and transmit the measurement signal to the first computing device. The sensor can be a capnometer, and the measurement signal can indicate a concentration of carbon dioxide exhaled by the patient. The sensor can be an oxygen sensor, and the measurement signal can indicate an amount of oxygen perfusion of the patient. The sensor can be a tissue perfusion sensor, and the measurement signal can indicate an amount of tissue perfusion of the patient. The sensor can be a brain oxygenation sensor, and the measurement signal can indicate an amount of brain oxygenation of the patient. The sensor can be a blood pressure sensor, and the measurement signal can indicate a blood pressure of the patient. The measurement signal can indicate one or more of an amount of oxygen perfusion, an amount of tissue perfusion, an amount of brain oxygenation, or a blood pressure of the patient.

Also disclosed by this document is a system including a first computing device and a chest compression device configured to communicate with the first computing device. The first computing device can be configured to obtain information regarding a patient being treated for cardiopulmonary arrest and to send commands to the chest compression device. The commands can include a defibrillator activation command to activate the defibrillator. The first computing device can further be configured to determine, from the patient information, a patient state indicative of cardiac dysrhythmia, wherein the first computing device, responsive to determining the patient state indicative of cardiac dysrhythmia, outputs to a display of the first computing device a recommendation to activate or deactivate the defibrillator.

In some implementations, the defibrillator can be configured to function as an external pacemaker.

In addition, the present disclosure covers methods that include: receiving, at a first computing device in a chest compression device including a defibrillator, a patient attribute signal measured by a patient sensor during operation of the chest compression device; determining, from the patient attribute signal, a patient state; responsive to determining the patient state, outputting to a display of the first computing device, a user prompt; receiving, as an input to the first computing device, a user command to activate the defibrillator; and responsive to receiving the user command to activate the defibrillator, transmitting a defibrillator activation command to the defibrillator.

In some implementations, the patient attribute signal can indicate one or more of an amount of oxygen perfusion, an amount of tissue perfusion, an amount of brain oxygenation, or a blood pressure of the patient.

The document also covers methods including: receiving at a first computing device, from a chest compression device including (a) a defibrillator and (b) an accelerometer configured to measure an acceleration of the chest compression device during operation of the chest compression device, the acceleration measured by the accelerometer; determining, from the accelerometer, a compression depth; and responsive to determining the compression depth, outputting to a display coupled to the first computing device a recommendation an indication of a compression quality.

Any of the above-described methods can further include receiving, at the first computing device and from a sensor, patient heart rate information; determining that the heart rate information is outside of a predetermined acceptable range of heart rates and/or heart rhythms; and responsive to determining that the heart rate information is outside of a predetermined acceptable range of heart rates and/or heart rhythms, activating the defibrillator.

Any of the above-described methods can further include, outputting, prior to activating the defibrillator, a warning that a defibrillation shock is about to be applied.

One or more of the implementations herein can provide various benefits. For example, in some cases, implementations of the electronic system can improve the likelihood of successfully treating a patient in cardiac arrest by allowing caretakers to coordinate or automate their efforts when treating the patient in cardiac arrest. The electronic system can provide each user with a clear indication of his role in an intervention team, provide information regarding what tasks to perform and when to perform those tasks, and provide feedback regarding the effectiveness of his performance. Further still, the electronic system can provide information to caretakers when treating a patient, without requiring that the caretakers hold a device, book, or other object while doing so. Still further, the electronic system can automate the performance of certain tasks and provide feedback regarding the effectiveness of the automated performance. Thus, the electronic system allows caretakers to treat the patient more effectively, and while having full use of their hands. Further, the electronic system can provide team management information to one or more users (e.g., a team leader), such that important information regarding the patient's care and the efforts of the users is readily accessible to an overseer. Further still, the electronic system can accurately record information regarding the patient's care, including the patient's vital signs, the tasks that were performed in treating the patient, and the time at which those tasks were performed. Thus, accurate records of the treatment can be subsequently reviewed during debriefings or other retrospective applications. Further still, the electronic system can be used as an education tool to instruct users in proper treatment techniques and protocols.

The details of one or more embodiments are set forth in the accompanying drawings and the description below. Other features and advantages will be apparent from the description and drawings, and from the claims.

### BRIEF DESCRIPTION OF THE DRAWINGS

FIG. 1A is a diagram of an example electronic system.

FIG. 1B is a diagram of an example of a system for performing cardiopulmonary resuscitation.

FIGS. 2A-2G are diagrams of examples of graphical user interfaces presented by a wearable computing device.

FIGS. 3A-3C are diagrams of examples of graphical user interfaces presented by a wearable computing device.

FIGS. 4A-4B are diagrams of examples of graphical user interfaces presented by a wearable computing device.

FIG. 4C is an examplary chart for determining a CPR quality index score.

FIG. 4D is an examplary chart for determining a perfusion quality index score.

FIG. 4E is an examplary chart showing the monitoring of the CPR quality index over a period of time.

FIGS. 5A-5C are diagrams of examples of graphical user interfaces presented by a wearable computing device.

FIG. 6 are diagrams of examples of graphical user interfaces presented by a wearable computing device.

FIG. 7 is a diagram of an example of a graphical user interface presented by a wearable computing device.

FIG. 8A is diagram of an example of a wearable computing device mounted on a compression plate through straps.

FIG. 8B-8C are diagrams of examplary swivel mechanism for mounting the wearable computing device shown in FIG. 8A to the straps.

FIG. 8D is a diagram of an example of grip or support for the compression plate shown in FIG. 8A.

FIG. 8E is a schematic illustrating a perspective view of an examplary chest compression device.

FIG. 8F is a schematic illustrating a top view of an exemplary chest compression device.

FIG. 8G is a schematic illustrating an exploded view of an exemplary chest compression device.

FIGS. 8H-8K are schematic illustrations of exemplary chest compression devices.

FIG. 8L is a block diagram illustrating an example of components of a chest compression device.

FIG. 8M is a schematic that illustrates an example of a chest compression device that includes a defibrillator.

FIG. 9A is a diagram of an example of a capnometer positioned on a bag valve mask.

FIG. 9B is a diagram of an example of a graphical user interfaces presented by a wearable computing device.

FIG. 10 is a diagram of examplary sensors for measuring aspects of a patient's condition.

FIG. 11A is a diagram of an examplary storage container.

FIG. 11B is a diagram of an examplary system for measuring the compression depth of a rescuer's chest compressions on a patient.

FIGS. 11C-11D are diagrams of an examplary system for measuring the compression depth of a rescuer's chest compressions on a patient.

FIG. 11E is a diagram of an examplary system for measuring the compression depth of a rescuer's chest compressions on a patient.

FIGS. 12A-12C are diagrams of examplary graphical user interfaces presented by a wearable computing device.

FIG. 13 is a flowchart diagram of an examplary process for assisting users in performing medical procedures.

FIG. 14 is a diagram of an examplary computer system.

FIG. 15 is a schematic illustrating an exemplary use of a combined chest compression and defibrillator device.

### DETAILED DESCRIPTION

When treating a patient in cardiac arrest, caretakers often follow guidelines for decision making and timing of interventions to improve the likelihood of success. In many cases, hospitals mandate that providers be trained in these life-saving protocols. Training can include, for example, classroom instruction and simulated scenarios.

However, real-life situations often do not resemble training scenarios. For example, during a training exercise, a complete team of caretakers is often present at the same time. Further, each member of the team is often pre-assigned a particular role within the team, and is already aware of the tasks that he needs to perform. Further still, there are often no extraneous personnel, alarms, or other barriers to effective communication between the team members.

In contrast, in a real-life situation, a patient's room is often filled with care providers, nurses, administrators, and students. If clear roles are not quickly established and a team leader does not assert him or herself, communication and treatment are often compromised. Further, team leaders are not always experienced in treating cardiac arrest. For example, in many cases, a team leader may be a physician who is generally relatively inexperienced in providing care (e.g., a first-year resident physician). Thus, the team leader may not be well versed in the proper treatment procedures.

Further, despite training, caretakers may suffer from information decay that can negatively impact their effectiveness in treating patients. For example, caretakers might undergo periodic training regarding the proper procedures in providing care. However, between training sessions, the caretakers may forget or misremember the procedures, particularly if a lengthy period of time has passed since the last training session. As a result, the caretakers' quality of treatment may suffer.

Further, in many cases, the use of technology is often poorly integrated into these scenarios. For example, medications given and procedures performed are often recorded on pen and paper by physicians and nurses, and immediate electronic decision-making support is often unavailable. As another example, time is often kept by an individual whose voice may be drowned out by other voices and noises in the room. Thus, after a medical intervention has concluded, there may be multiple—sometimes inaccurate—records of when interventions occurred, which affects the quality of debriefings.

To improve the likelihood of success, caretakers can use an electronic system to coordinate their efforts when treating a patient in cardiac arrest. The electronic system can provide each team member with a clear indication of the team member's role in an intervention team, provide information regarding what tasks to perform and when to perform those tasks, and provide feedback regarding the effectiveness of the team member's performance. Further still, the electronic system can provide information to caretakers when treating a patient, without requiring that the caretakers hold a device, book, or other object while doing so. Thus, the electronic system allows caretakers to treat the patient more effectively, and while having full use of their hands.

Further, the electronic system can provide team management information to one or more users (e.g., a team leader), such that important information regarding the patient's care and the efforts of the users is readily accessible to an overseer. For example, the electronic system may help administrators assess a team member's compliance with established treatment protocols, and to evaluate the performance of one or more team members during and after their efforts in treating a patient. Further still, the electronic system can accurately record information regarding the patient's care, including the patient's vital signs, the tasks that were performed in treating the patient, and the time at which those tasks were performed. Thus, accurate records of the treatment can be subsequently reviewed during debriefings or other retrospective applications. Further still, the electronic system can be used as an education tool to instruct team members in proper treatment techniques and protocols.

Still further, the electronic system can automate the performance of certain tasks (e.g., administration of chest compressions and/or defibrillating shocks) and provide real-time feedback regarding the effectiveness of the automated tasks performed. Thus, caretakers can monitor and treat the patient more effectively, without the distraction of manually performing chest compressions or administering defibrillating shocks.

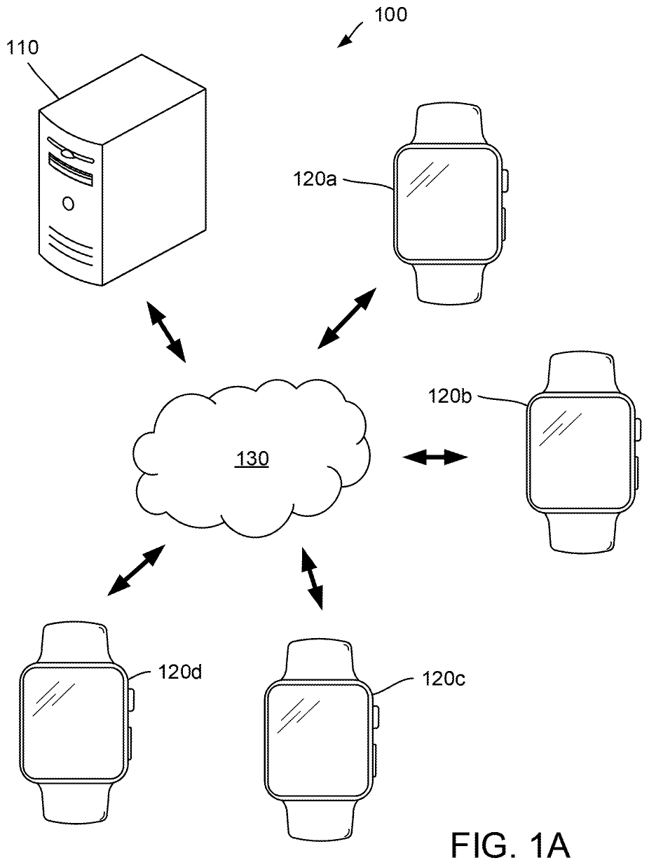

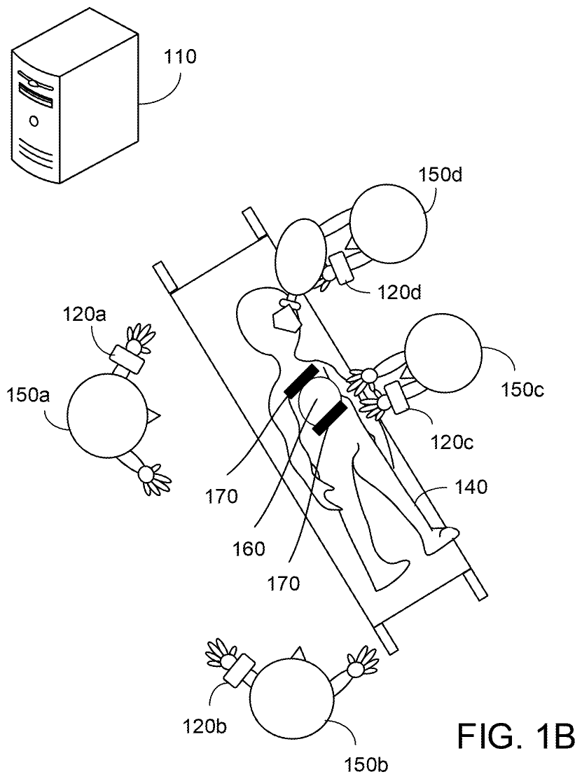

An example electronic system 100 is shown in FIG. 1A. An example environment for utilizing the electronic system 100 is shown in FIG. 1B. In the example environment shown in FIG. 1B, a patient 140 is in cardiac arrest and is being treated by four members 150a-d of a medical intervention team. Each of the members 150a-d is assigned a particular role in treating the patient. For example, the member 150a is assigned a leadership role, and is tasked with coordinating and managing the efforts of the other members of the team in treating the patient. In another example, the member 150b is assigned the recorder role, and is tasked with recording the tasks that were performed during the course of treating the patient. In another example, the member 150d is assigned a respiration monitoring role, and is tasked with monitoring the respiration of the patient during the course of treatment and administrating breaths as necessary.

In another example, the member 150c is assigned a compression role, and is tasked with monitoring and/or applying compressions to the patient during the course of treatment. The compressions may be applied using a chest compression device 160. The chest compressions may be applied automatically by device 160, without human intervention to cause the application of force, or the chest compressions may be applied manually using the device 160. In some implementations, the member 150c also may be tasked with monitoring and/or applying defibrillations to the patient during the course of treatment. In such cases, the defibrillations may also be applied using the chest compression device 160, which may optionally include a defibrillator component 170. The defibrillations may be applied automatically by device 170, without human intervention to trigger the application of electric potential to the patient, or the defibrillations may be applied manually using the device 170, such that the user triggers when the electric potential is applied.

Although example roles are described above, these are merely an illustrative example. In practice, other roles are also possible, either instead of in addition to those described above. Alternatively, or in addition, users may combine roles. For example, the rescuer 150d can be tasked with performing cardiopulmonary resuscitation (CPR) (e.g., applying chest compressions to the patient) and/or administering defibrillating shocks during the course of treatment in addition to monitoring the respiration of the patient during the course of treatment and/or administrating breaths as necessary.

Each member 150a-d can wear a respective wearable computing device 120a-d. Each wearable computing device 120a-d is programmed to assist its wearer in performing his duties as a part of the intervention team, as described in the examples below. Although certain functions are described herein as being performed by particular wearable computing device, any of the wearable devices may be configured to perform some or all of the described functions.

For instance, in some implementations, the wearable computing device 120a can be programmed to assist the member 150a in performing his duties as a leader of the team. For example, the wearable computing device 120a can collect information from each of the other wearable computing devices 120b-d, and present the information to the member 150a to assist him in managing the effort of the intervention team. The wearable computing device 120a can also transmit information to each of the other wearable computing devices 120b-d to assist the member 150a in directing efforts of each member of the team. The wearable computing device 120a can also receive information from the computing device 110 (e.g., information regarding the patient, such as patient records, or information regarding potential treatment protocols that can be used to treat the patient). The wearable computing device 120a can also transmit information to the computing device 110 (e.g., information regarding the actions taken by the member 150a, and information received from each of the other wearable computing devices). Members 150a-d can operate the wearable computing devices 120a-d using an interactive display on the wearable computing devices 120a-d or through voice commands. Any of the other wearable computing devices 120b-120d also may be configured to perform the same functions as described herein for wearable computing device 120a.

In some implementations, the wearable computing device 120b can be programmed to assist the member 150b in performing his duties as the recorder for the team. For example, the wearable computing device 120b can present the member 150b with a series of tasks that can be potentially performed by the members of the team, and allow the member 150b to specify which of those specific tasks that have been performed. The wearable computing device 120b can also record information regarding the performance of those tasks (e.g., the time at which the task was performed, performance parameters associated with those tasks, and so forth). Performance parameters can include, for example, details regarding how a particular task was performed. As an example, if a defibrillation was performed, a performance parameter can include the amount of energy that was applied in performing the defibrillation, the voltage applied during the defibrillation, the current applied during the defibrillation, or the pulse duration associated with the defibrillation. As another example, if a compression was performed, a performance parameter can include a depth of the compression (the depth that the chest is compressed), a pressure applied during the compression, the compression frequency, whether the compression depth is meets a predefined compression depth (e.g., about 1.5 inches for infants, within about 2 and 2.4 inches for children and adults), whether the pressure applied is within a predefined pressure range, or whether the compression frequency is within a predefined range (e.g., within about 100 to 200 compressions per minute). As another example, if a drug was administered, performance parameters can include the type of drug administered, the amount of drug administered, the rate at which it was administered, and/or other information pertaining to the administration of the drug. This information can in some cases, be recorded automatically by the wearable computing device 120b, or in some cases, manually entered by its wearer. The wearable computing device 120b can also transmit this information to the wearable computing device 120a (e.g., to provide the member 150a with information to assist him in making decisions on behalf of the team) and/or the computing device 110 (e.g., to record the information to future retrieval and review). In some cases, information obtained by the wearable computing device 120b can be used to modify the behavior of other devices of the electronic system 100 (e.g., resetting countdown timers and/or displaying alerts on one or more of the other devices).

If one or more members are assigned to a rescuer role, a wearable computing device 120c can be programmed to assist the rescuer 150c in performing his duties as the rescuer 150c. For example, the wearable computing device 120c can present the rescuer 150c with instructions from the member 150a and instruct the rescuer 150c to perform a particular action with respect to the patient (e.g., applying chest compressions or defibrillating shocks to the patient). The wearable computing device 120c can also obtain sensor data, and based on that sensor data, determine information regarding the efforts of the rescuer 150c. For example, the wearable computing device 120c can acquire sensor information from pressure sensors, accelerometers, gyroscopes, chest displacement sensors, and/or other motion sensors, and determine the length of time that the rescuer has been performing chest compression or administering defibrillating shocks, the rate at which the rescuer is preforming chest compressions or administering defibrillating shocks, the pressure or force being applied to the patient by those chest compressions, the depth of the chest compressions, or the potential applied by the defibrillating shocks, among other information. The wearable computing device 120c can also present that information to the rescuer 150c, such that the rescuer 150c can adjust his performance accordingly. The wearable computing device 120c can also transmit this information to the wearable computing device 120a (e.g., to provide the member 150a with information to assist him in making decisions on behalf of the team), other computing devices (e.g., 120b and 120d), and/or to the computing device 110 (e.g., to record the information to future retrieval and review).

The wearable computing device 120d can be programmed to assist the member 150d in monitoring the respiration of the patient during the course of treatment. For example, the wearable computing device 120d can also obtain sensor data, and based on that sensor data, determine information regarding the respiration of the patient 140. For example, the wearable computing device 120d can acquire sensor information from oxygen sensors, carbon dioxide sensors, perfusion sensors, and/or other sensors, and determine information regarding the respiration and perfusion of the patient from the data obtained by those sensors. The wearable computing device 120d can also present that information to the member 150d, such that the member 150d can adjust this performance accordingly (e.g., by increasing the delivery of oxygen to the patient). The wearable computing device 120d can also transmit this information to the wearable computing device 120a (e.g., to provide the member 150a with information to assist him in making decisions on behalf of the team), to the other computing devices (e.g., 120b and 120c), and/or the computing device 110 (e.g., to record the information to future retrieval and review).

The functionality of each of the components of the system 100 is described in greater detail below.

In general, the computing device 110 can be any electronic device that processes, transmits, and receives data. The computing device 110 stores information relating to the patient's treatment. For example, the computing device 110 can store information regarding the performance of particular tasks during the patient's treatment and the time in which those tasks were performed. In some cases, the computing device 110 can also manage communications between the wearable computing devices 120a-d. For example, in some cases, the computing device 110 can receive information from one or more of the wearable computing devices 120a-d, and transmit some or all of that information to one or more of the other wearable computing devices 120a-d. In some cases, the computing device 110 can also transmit information collected from each of the wearable computing devices 120a-d to other devices for recordation (e.g., by exporting the information or a summary of the information to an electronic medical records system). Examples of the computing device 110 include computers (such as desktop computers, notebook computers, server systems, embedded devices, etc.), mobile computing devices (such as cellular phones, smartphones, tablets, personal data assistants, notebook computers with networking capability), and other computing devices capable of transmitting and receiving data from network 130. In some cases, the computing device 110 can be remote from the wearable computing devices 120a-d. For example, in some cases, the computing device 110 can be one or more server computers located in a different room, building, or a geographical region than those of the wearable computing devices 120a-d. The computing device 110 can include devices that operate using one or more operating system (e.g., Microsoft Windows, Apple OSX, Linux, Unix, Android, Apple iOS, Apple watchOS, etc.) and/or architectures (e.g., x86, PowerPC, ARM, etc.).

The wearable computing devices 120a-d can be any electronic device that can be worn on a user's body that processes, transmits, and receives data. Examples of the wearable computing devices 120a-d include devices that can be worn on a user's wrist (e.g., a “smart watch”), devices that can be worn over a user's eye (e.g., “smart glasses”), or devices that can be worn on other parts of a user's body (e.g., hands, arms, head, etc.). In some cases, the wearable computing devices 120a-d can be releasably secured to one or more of recovery devices used during a CPR procedure. For instance, the wearable computing device 120c can be releasably fixed to the compression device 160 (which may include the defibrillator 170). Though described in the present disclosure as “wearable,” in some implementations, one or more of the computing devices 120a-d are not wearable. Rather, the one or more computing devices 120a-d may include, e.g., a mobile computing device such as a smart phone or a computing tablet that can perform any and all of the same functions and operations as the wearable computing devices described herein. In some implementations, one or more of the computing devices 120a-d may be fixed to and inseparable from a recovery device. For instance, the computing device 120c can be fixed to the compression device 160 and/or to the defibrillator 170 without being configured to be detached by a user. The wearable computing devices 120a-d can each contain one or more electronic control modules (e.g., combinations of circuitry, firmware, and/or software) that allow each wearable computing device 120a-d to receive, interpret, process, and transmit information. The wearable computing devices 120a-d can include devices that operate using one or more operating system (e.g., Microsoft Windows, Apple OSX, Linux, Unix, Android, Apple iOS, Apple watchOS, etc.) and/or architectures (e.g., x86, PowerPC, ARM, etc.).

In some cases, wearable computing devices 120a-d present information to its wearer (e.g., through a screen or other display device), and allow the wearer to input selections, commands, or other inputs (e.g., through a touch sensitive surface, buttons, dials, knobs, levels, switches, and so forth). In some cases, a wearable computing device 120a-d can include a touch sensitive screen that both displays information to a wearer and allows the user to input information into the wearable computing device 120a-d by touching the screen. In some cases, a wearable computing device can include a microphone that allows the user to input information by speaking words or phrases. Examples of information that may be entered into computing devices 120a-d includes, among other things, patient status (such as whether a patient intervention, e.g., intubation, intravenous line placement, intra osseous access, sonogram, or pericardiocenthesis, is being performed) as well as time stamps when a specified activity takes place (such as when an intervention is begun).

In some cases, the wearable computing devices 120a-d can communicate with other computing devices 120a-d and/or the computing device 110 directly (e.g., directly over a communications network). In some cases, the wearable computing devices 120a-d can communicate with other computing devices 120a-d and/or the computing device 110 indirectly. As an example, one or more of the wearable computing devices 120a-d can be “paired” to a respective mobile device (e.g., via a Bluetooth connection, near field communication (NFC) connection, or some other network connection to a cellular phone, a smart phone, a tablet, or some other mobile device). That mobile device can then communicate with another wearable computing device 120a-d, either directly with that wearable computing device 120a-d, or indirectly through another mobile device “paired” to that wearable computing device 120a-d. Similarly, one or more of the wearable computing devices 120a-d can be “paired” to one or more separate and discrete sensors (e.g., via a Bluetooth connection, near field communication (NFC) connection, or some other network connection to one or more sensors).

The communication network through which the computing devices 120a-d, the computing device 110, and optionally the chest compression device 160 and the defibrillator 170 can include any communications network through which data can be transferred and shared. For example, network 130 can be a local area network (LAN) or a wide-area network (WAN), such as the Internet. The network can be implemented using various networking interfaces, for instance wireless networking interfaces (such as WiFi, Bluetooth, or infrared) or wired networking interfaces (such as Ethernet or serial connection). The network also can include combinations of more than one network, and can be implemented using one or more networking interfaces.

As described above, each of the wearable computing devices 120a-d can be programmed to assist a user. For example, the wearable computing device 120a can be programmed to assist a user assigned to the leader role (e.g., member 150a, also referred to as the “leader”). In some cases, the wearable computing device 120a can receive patient information from the computer device 110 or another external system (e.g., a patient intake system, a hospital notification system, an emergency code notification system, or an electronic medical records system). Based on this information, the wearable computing device 120a can notify the leader that a patient is in cardiac arrest, the location of that patient (e.g., a particular room or area of a hospital), and/or the time at which the cardiac arrest began.

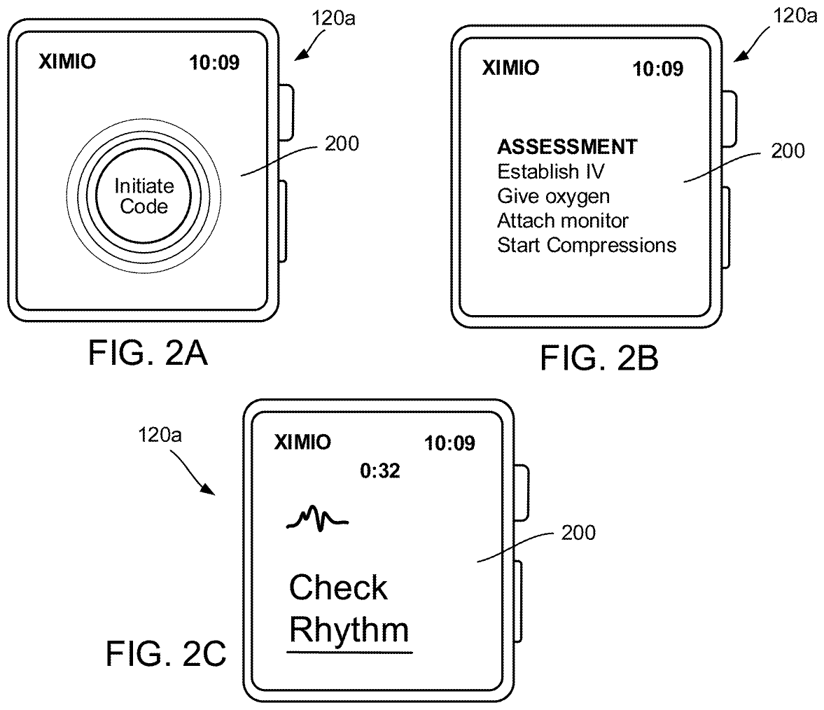

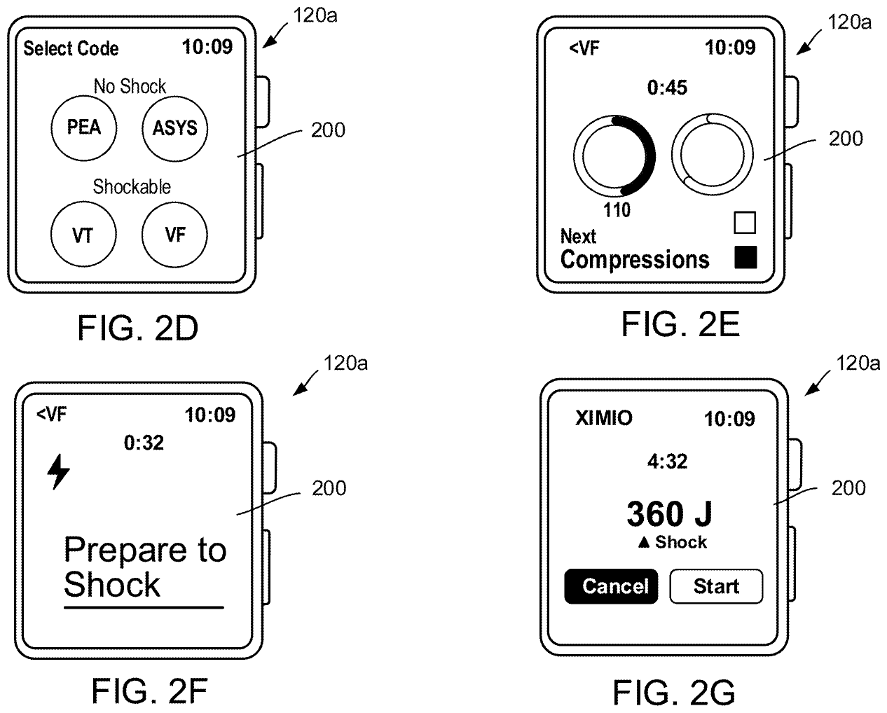

In some implementations, the wearable computing devices 120a-d allow the leader to initiate a medical intervention on behalf of the patient. For example, as shown in FIG. 2A, the wearable computing device 120a can include a graphical user interface (GUI) 200 that presents the leader with an option to initiate a medical intervention (often referred to as initiating a “code”). When the leader selects this option (e.g., by touching an icon on a screen of the device or by giving an audible command to the device), the wearable computing device 120a records the time at which the selection was made (e.g., by recording a timestamp), and transmits this information to the computing device 110 for storage. The wearable computing device 120a and/or the computing device 110 can also transmit a notification to one or more of the other wearable computing devices 120b-d to inform their members of the intervention team that their services are needed.

As shown in FIG. 2B, after the leader initiates the intervention, the wearable computing device 120a presents the leader with a list of possible intervention protocols that an intervention team can follow to treat the patient. The possible intervention protocols can include the automatic application of chest compressions and/or defibrillating shocks by the chest compression device 160 and the defibrillator 170. Information regarding each protocol can be retrieved, for example, from the computing system 110, and/or pre-stored on some of all of the wearable computing devices 120a-d. Each protocol can include, for example, specific tasks to be performed with respect to the patient, particular conditions under which to perform those tasks (e.g., at a particular carbon dioxide concentration (or range thereof) exhaled by the patient, at a particular oxygen perfusion (or range thereof) of the patient, at a particular tissue perfusion (or range thereof) of the patient, at a particular blood pressure (or range thereof) of the patient, at a particular brain oxygenation (or range thereof) of the patient, at a particular pulse pressure (or range thereof) or pulse waveform (or range thereof) of the patient), whether the tasks are to be performed manually (i.e., by a person on the intervention team) or automatically (e.g., by the chest compression device 160 and/or the defibrillator 170), and times in which to perform those tasks. Each protocol can also specify, for example, the division of tasks between multiple members of an intervention team. For example, each protocol can specify that particular tasks be performed by particular members of the team, while other tasks be performed by other members of the team. In some cases, each protocol can be specific for treating a particular medical diagnosis, and the leader can select an appropriate protocol based on his assessment of the patient. When the leader selects a particular protocol, the wearable computing device 120a records the time at which the selection was made (e.g., by recording a timestamp), and transmits this information to the computing device 110 for storage.

Upon selecting a protocol, the wearable computing device 120a presents the leader with one or more prompts to perform tasks in accordance with the selected protocol. For example, the selected protocol may specify that the cardiac rhythm of the patient be measured at a particular time; thus, as shown in FIG. 2C, the wearable computing device 120a can present the leader with a prompt to measure the cardiac rhythm of the patient at the appropriate time. The prompt can include, for example, a visual indication (e.g., an image, text, an animation, a “pop-up,” a change in color, or other visual indication), an audible indication (e.g., a sound effect, music, or other audible indication), and/or a haptic indication (e.g., a vibration).

Further, electronic system 100 can prompt other users to perform tasks in accordance with the selected protocol. For example, subsequent to the selection of a particular protocol by the leader, information about the selected protocol can be provided to one or more of the wearable computing devices 120a-d by, for example, the computing device 110 and/or by the wearable computing device 120a. The information provided to the one or more wearable mobile devices can include, e.g., one or more particular tasks for a user perform during the intervention and the time at which such tasks should be performed. For example, a user assigned to the recorder role (e.g., member 150b, also referred to as the “recorder”) is wearing the wearable computing device 120b (programmed to assist those assigned to the recorder role); thus, the wearable computing device 120b can present the recorder with a prompt to perform a particular task at the appropriate time. Similarly, a user assigned to the rescuer role (e.g., member 150c, also referred to as the “rescuer”) is wearing the computing device 120c (programmed to assist those assigned to the rescuer role); thus, the wearable computing device 120c can present the rescuer with a prompt to perform a particular task (e.g., perform a compression or perform a defibrillation) assigned to the rescuer at the appropriate time.

Alternatively, the wearable computing device 120a can send a command to the compressor 160 to automatically begin compressions. For instance, the command may be sent wirelessly through network 130 to the compressor device 160. The compression command may include information for performing the compression including details such as depth of compression, compression rate, number of compressions, time period during which compressions are applied, and pressure to apply to achieve a specified depth of compression. Upon receiving the compression command, the compression device 160 may automatically begin performing compressions. In some implementations, the wearable computing device 120a (and/or another wearable computing device, such as computing device 120c) may record additional information relevant to the compression, such as the time at which the selection was made (e.g., by recording a timestamp) and information regarding the performance of the task (e.g., the depth of the compression). The recorded information then may be transmitted to the computing device 110 for storage. In some cases, this information can be presented by one or more of the other wearable computing devices 120a-d. For example, in some cases, the wearable computing device 120b can also present an interface to the member 150b (e.g., the recorder), such that he can also confirm performance of the task. Similarly, upon confirmation, the wearable computing device 120b records the time at which the selection was made and information regarding the performance of the task, and transmits this information to the computing device 110 for storage.

Likewise, a user assigned to the respiration monitoring role (e.g., the member 120d, or the “respiration monitor”) is wearing the computing device 120d (programmed to assist those assigned to the respiration monitoring role); thus, the wearable computing device 120d can present the respiration monitor with a prompt to perform a particular task assigned to the recorder at the appropriate time.

In some cases, the selected protocol may specify different courses of treatment depending on the condition of the patient at a particular time. In some cases, information regarding the condition of the patient can be determined based on the assessment by the leader. For example, as shown in FIG. 2D, the wearable computing device 120a can present the member 150a with several different diagnostic choices, each corresponding to a different possible condition of the patient. The leader can select an appropriate diagnosis based on his assessment of the patient. When the leader selects a particular diagnosis, the wearable computing device 120a records the time at which the selection was made (e.g., by recording a timestamp), and transmits this information to the computing device 110 for storage.

In some cases, a wearable computing device can alert a user that a particular task should be taken in the future, such that the user is prepared to perform that task at the appropriate time. For example, when the member 150a selects the “VF” diagnosis shown in FIG. 2D (corresponding to a diagnosis of ventricular fibrillation), in response, the wearable computing device 120a can present information to the member 150a regarding a protocol to be performed based on that diagnosis. For instance, the protocol may specify that the leader defibrillate a patient at a particular time; as shown in FIG. 2E, the wearable computing device 120a can present a timer 202 that counts down (e.g., via a numerical countdown and/or an animated progress bar or arc), indicating when the defibrillation should be performed.

As shown in FIG. 2F, when the countdown is complete, the wearable computing device 120a presents an alert to the member 150a that defibrillation should be performed at that time. Alternately, the wearable computing device 120a can send a command to the defibrillator 170 to automatically deliver a defibrillating shock to the patient at that time. For instance, the command may be sent wirelessly through network 130 to defibrillator 170 that is formed as a component coupled to the chest compression device 160.

As shown in FIG. 2G, the wearable computing device 120a can subsequently present information regarding the defibrillation (e.g., the suggested energy that should be applied to the patient), and an option to perform a next defibrillation (e.g., a “start” button). When the member 150a selects the option to perform the defibrillation, the wearable computing device 120a may transmit a defibrillation command to the defibrillator 170. The defibrillation command may include information for performing the defibrillation including details such as energy, voltage, and/or duration of the defibrillation charge to be applied to the patient. Upon receiving the defibrillation command, the defibrillation device 170 may automatically begin performing defibrillation. In some implementations, the wearable computing device 120a may record additional information relevant to the defibrillation, such as the time at which the selection was made (e.g., by recording a timestamp) and information regarding the performance of the task (e.g., the energy of the defibrillation). The recorded information then may be transmitted to the computing device 110 for storage. In some cases, this information can be presented by one or more of the other wearable computing devices 120a. For example, in some cases, the wearable computing device 120b can also present an interface (e.g., the interface shown in FIG. 2G) to the member 150b (e.g., the recorder), such that he can also confirm performance of the task. Similarly, upon confirmation, the wearable computing device 120b records the time at which the selection was made and information regarding the performance of the task, and transmits this information to the computing device 110 for storage.

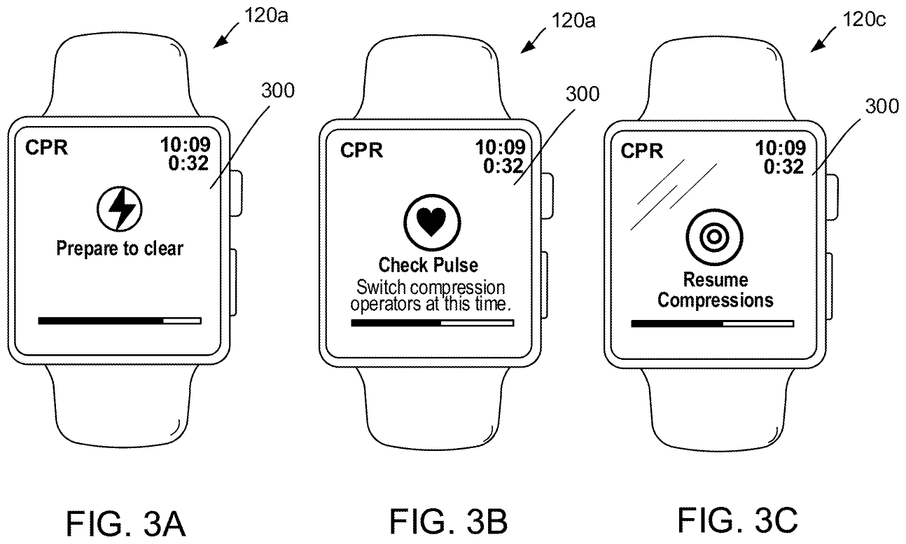

Although example tasks are described above, these are merely illustrative examples. In practice, a protocol can specify other tasks, either instead of or in addition to those described above. Accordingly, the wearable computing device 120a can provide other information to the leader, as appropriate for the particular task. Further, although particular tasks are described as being assigned to the leader, in practice, these tasks can be assigned to other members of the intervention team. Accordingly, the other wearable computing devices 120b-d can provide information to their respective users, as appropriate for the particular task. To illustrate, FIGS. 3A-C show additional example alerts and/or prompts that can be displayed to one or more members of the intervention team in accordance with the selected protocol. FIG. 3A shows an example wearable computing device 120a and GUI 300 displaying an alert to the leader to prepare to clear the patient in preparation for defibrillation. FIG. 3B shows an example wearable computing device 120a and GUI 300 displaying a prompt to the leader to check the patient's pulse. FIG. 3C shows an example wearable computing device 120c and GUI 300 displaying a prompt to the rescuer to resume performing CPR on the patient. In some implementations, the wearable computing device 120a can transmit a command to the chest compression device 160 to automatically begin or resume chest compressions. In some cases, the command that is transmitted to the chest compression device 160 may include information relevant to performing the chest compression such as depth of compression, force, pressure, duration of compression, and/or number of compressions to be performed. In some cases, the wearable computing device 120a may present the user with an option to trigger the transmission of the command to begin automatic chest compressions, similar to the start button for commencing automatic defibrillation shown in FIG. 2G. In some implementations, the wearable computing device 120c can send a command to the chest compression device 160 to resume performing chest compressions on the patient after an initial chest compression has been performed and/or interrupted. In practice, the wearable computing devices can present other alerts, prompts, or information, depending on the implementation.

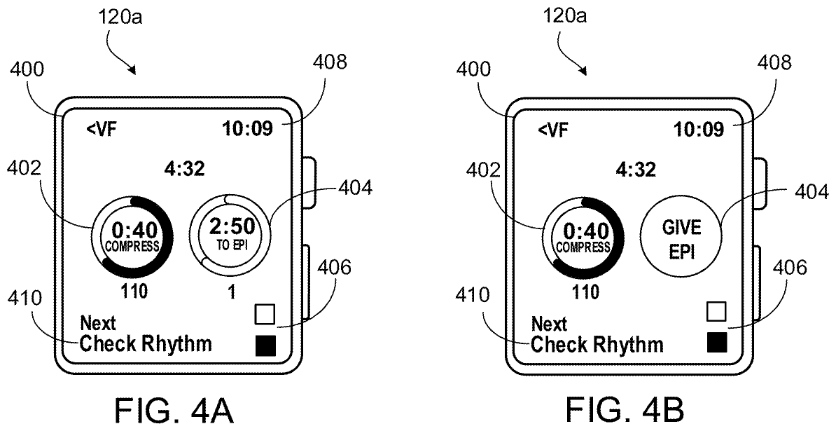

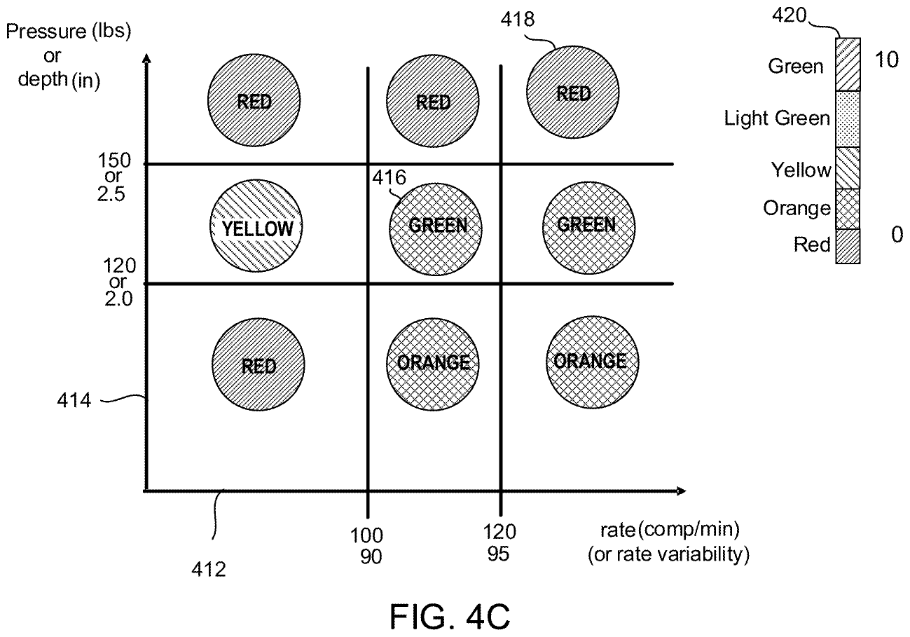

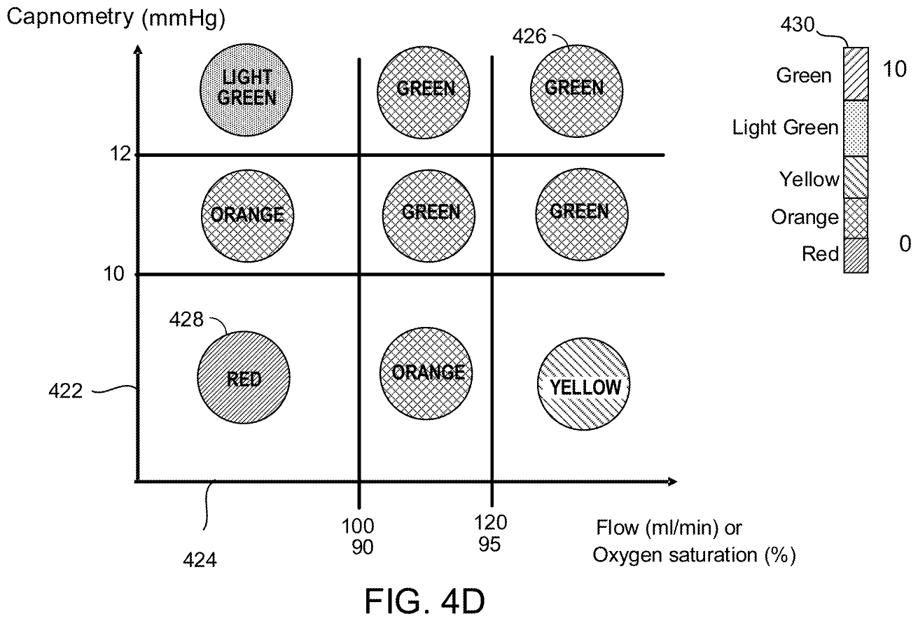

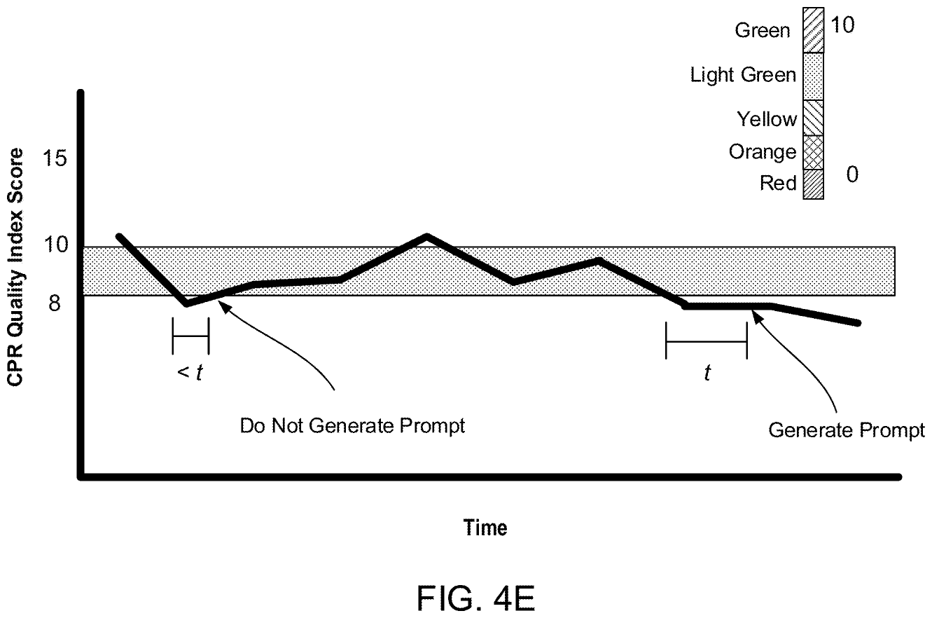

In some implementations, the wearable computing device 120a can provide the leader with information that summarizes the patient's condition, the task or tasks currently being performed, and future tasks or tasks to be performed. As an example, as shown in FIG. 4A, the wearable computing device 120a can present a GUI 400 with a CPR portion 402. The CPR portion 402 includes a timer that indicates a recommended amount of time that a rescuer should continuously perform CPR (e.g., according to the selected protocol or general guidelines), and a length of time that the current rescuer has been performing CPR on the patient. When the timer expires, this indicates that a different rescuer should take over performing CPR on the patient. In response, the system 100 can notify another user to perform CPR. Alternately, or if no other users begin performing CPR, the system 100 can send an electronic command to the chest compression device 160 to automatically perform chest compressions on the patient if the chest compression device 160 is configured to perform automatic chest compressions. The electronic command can be sent to the chest compression device responsive to the timer expiring or, alternatively, the wearable computing device 120a can display an activation command (e.g., a “start” button”) responsive to the timer expiring that allows the leader to select activation of the automatic chest compression by the chest compression device 160. In some implementations, the wearable computing device 120a can display a notification to the member 150a, reminding the leader to instruct another member of the team to perform CPR. As another example, in some cases, the wearable computing device 120c can display a notification to the rescuer, reminding the rescuer to cease performance of CPR and pass the duties to another member of the team. This can be beneficial, for example, to reduce the likelihood that a rescuer becomes fatigued in performing CPR for too long a period of time. The wearable computing device 120c can be configured to display such notifications in circumstances when the compression deviates from a predetermined or ideal compression. For instance, if the compression rate falls below a predetermined compression rate and/or the chest compression pressure/force being applied falls below a predetermined pressure/force, the wearable computing device 120c may output to a display a recommendation (e.g., a text notification) to have chest compressions performed by a less fatigued member of the team. Alternatively, or in addition, the wearable computing device 120c may display such notifications when the pressure decay, compression depth, and/or compression rate variability deviates from a predetermined or ideal pressure decay, compression depth, and/or compression rate variability, respectively. In some implementations, the CPR portion 402 may output to a display the compression rate or force/pressure of the CPR so that the team member can view the performance of the chest compression. Determination of the compression rate is described in greater detail below.

In some implementations, the wearable computing device 120a also presents a GUI 400 having an upcoming task portion 404. The upcoming task portion 404 indicates an upcoming task that is to be performed in accordance with the selected protocol, and a timer that counts down the time at which that task should be performed. As shown in FIG. 4B, when the timer expires, the wearable computing device 120 can provide a prompt in the upcoming task portion 404 to perform the task. Alternatively, in some implementations, when the timer expires, the wearable computing device 120a may issue an electronic command to a device for performing the upcoming task, such as a chest compression device or a defibrillator.

In some implementations, the wearable computing device 120a also presents a GUI 400 having a task preview portion 410. The task preview portion 410 indicates an upcoming task that is to be performed after upcoming task in accordance with the selected protocol (e.g., the task to be performed subsequent to that shown in the upcoming task portion 404).

In some implementations, the wearable computing device 120a also presents a GUI 400 having a treatment status portion 406. The treatment status portion 406 indicates the quality and/or efficacy of the intervention team's treatment of the patient. For example, the treatment status portion 406 can include an indication of a CPR quality index (e.g., a composite index based on factors such as compression rate, compression pressure, compression release pressure, pressure decay, compression depth, and/or compression rate variability) and/or a perfusion quality index (e.g., a composite index based on factors such as perfusion pressure, arterial pressure and/or end tidal CO, and/or brain/tissue saturation). Perfusion quality index may be computed, e.g., based on measurements received, in part, from a perfusion sensor that is a component part of the chest compression device or a separate sensor applied to a different area of the patient from the chest compression device. These indications can be provided as a numerical score (e.g., on a quality scale of 1-10) and/or as a color score (e.g., on a quality scale of red, orange, yellow, light green, and green, indicating an ascending quality scale from “poor” to “good” quality). The GUI 400 can also include a time portion 408 that indicates the time that has elapsed since the leader initiated the intervention. Alternately or in addition, if the intervention team is treating the patient manually and the CPR quality index is assigned a “poor” or “fair” score, the wearable computing device 120a can transmit a command to the chest compression device 160 to begin performing automatic chest compressions on the patient and/or a command to the defibrillator 170 to begin automatically administering defibrillating shocks to the patient.

An example technique for determining a score for the CPR quality index is shown in FIG. 4C. As shown in FIG. 4C, two factors can be considered in determining a CPR quality index score: compression rate (shown on axis 412) and compression pressure (shown on axis 414). As an alternative to compression pressure, compression depth may be used. Depending on the values of the compression rate and compression pressure, a particular qualitative score can be assigned. For example, when the compression pressure is between 120 and 150 lbs. and the compression rate is between 100 and 120 compressions per minute, the CPR quality index can be assigned a “good” score (e.g., depicted as a green colored icon 416). However, when the compression pressure is greater than 150 lbs. and the compression rate is greater than 120 compressions per minute, the CPR quality index can be assigned a “poor” score (e.g., depicted as a red colored icon 418), indicating that the rescuer is performing compressions in an ineffective or unsafe manner. Further, when the compression pressure is less than 120 lbs. and the compression rate is greater than 100 compressions per minute, the CPR quality index can be assigned a “fair” score (e.g., depicted as a orange colored icon 420), indicating that the rescuer is performing compressions moderately effectively. Likewise, other combinations of values can correspond to different scores for the CPR quality index. Although example ranges for each factor are provided, these are merely illustrative examples. In practice, other ranges are possible, depending on the implementation. Further, although example “color” scores are described, these are merely illustrative examples. In practice, any number of colors can be used to represent scores (e.g., two colors, three colors, four colors, and so forth). Likewise, numerical scores can be used either instead of, or in combination, with color scores. As an example, as shown in FIG. 4C, a color scale 420 can be used to represent both color scores and numerical scores across a range.

Although two example variables are described above, those are merely illustrative examples. In practice, different variables can be used to determine a score. For example, as shown in FIG. 4C, instead of determining a score based on compression rate, compression depth can be used instead (e.g., as shown on axis 414). As another example, as shown in FIG. 4C, instead of determining a score based on compression rate, compression rate variability can be used instead (e.g., as shown on axis 412). Compression variability can be, in some cases, the number of deviations from a particular range of compression rates (e.g., an ideal or recommended range of compression rates. Further, although a two-variable technique for determining a score is described, this is merely an illustrative example. In practice, any number of different variables can be used to determine a score (e.g., a three-variable technique, a four-variable technique, etc.).

This technique can be similarly used to determine score of the perfusion quality index. As an example, a technique for determining a score for the perfusion quality index is shown in FIG. 4D. As shown in FIG. 4D, two factors can be considered in determining a score: carbon dioxide concentration obtained via capnometry (shown on axis 422) and vascular flow rate (shown on axis 424). Depending on the values of the carbon dioxide concentration and vascular flow rate, a particular qualitative score can be assigned. For example, when the carbon dioxide concentration is greater than 12 mmHg and the vascular flow rate is greater than 120 ml/min, the perfusion quality index can be assigned a “good” score (e.g., depicted as a green colored icon 426). However, when the carbon dioxide concentration is less than 10 mmHg and the vascular flow rate is less than 100 ml/min, the perfusion quality index can be assigned a “poor” score (e.g., depicted as a red colored icon 428), indicating that rescue breathes are being administered in an ineffective manner. Likewise, other combinations of values can correspond to different scores for the perfusion quality index. Although example ranges for each factor are provided, these are merely illustrative examples. In practice, other ranges are possible, depending on the implementation. As an example, in some cases, the ranges of vascular flow rate can be determined based on the location at which the vascular flow is being measured. For instance, when the average vascular flow rate in a healthy patient's leg is 284±21 ml/min in the common femoral (CFA), 152±10 mL/min in the superficial femoral (SFA), 72±5 mL/min in the popliteal, and 3±1 mL/min in the dorsalis pedis. Thus, the range of vascular flow rates for each score can be adjusted to account for differences in the measurement site. Alternately or in addition, if the intervention team is treating the patient manually and the perfusion quality index is assigned a “poor” or “fair” score, the wearable computing device 120a can send a command to the chest compression device 160 to begin performing chest compressions on the patient and/or a command to the defibrillator 170 to begin administering defibrillating shocks to the patient.

Further, although example “color” scores are described, these are merely illustrative examples. In practice, any number of colors can be used to represent scores (e.g., two colors, three colors, four colors, and so forth). Likewise, numerical scores can be used either instead of, or in combination, with color scores. As an example, as shown in FIG. 4D, a color scale 430 can be used to represent both color scores and numerical scores across a range.

Similarly, although two example variables are described above, those are merely illustrative examples. In practice, different variables can be used to determine a score. For example, as shown in FIG. 4D, instead of determining a score based on vascular flow rate, the percentage of oxygen saturation can be used instead (e.g., as shown on axis 424). Further, although two a two-variable technique for determining a score is described, this is merely an illustrative example. In practice, any number of different variables can be used to determine a score (e.g., a three-variable technique, a four-variable technique, etc.).

In some cases, the scores can be used to determine when a rescuer is not effectively or safely performing his rescue tasks, and in response, prompt one of the members of the intervention team to take corrective action, and/or send a command to the chest compression device 160 and/or or the defibrillator 170 to take corrective action (e.g., begin automatically administering chest compressions to the patient and/or begin automatically administering defibrillating shocks to the patient). For instance, in some cases, the system 100 can notify another user to take over the task of performing CPR. As an example, in some cases, the wearable computing device 120a can display a notification (e.g., a text message or graphic) to the member 150a, suggesting that the leader instruct another member of the team to perform CPR. As another example, in some cases, the wearable computing device 120c can display a notification (e.g., a text message or graphic) to the rescuer, suggesting that the rescuer cease performance of CPR and pass the duties to another member of the team. As another example, in some cases, the wearable computing devices 120a and/or 120c can present information its wearer with instructions for improving his performance (e.g., a prompt to slow down or speed up his compressions, and/or to apply more or less pressure during his compressions).

In some cases, the system 100 can determine that a rescuer is not effectively or safely performing his rescue tasks when a score drops below a threshold level for a particular period of time. For example, as shown in FIG. 4E, the system 100 can record the scores for the CPR quality index over a period of time. If the score drops below a threshold level (e.g., a score of 8) for a threshold length of time t or longer (e.g., 5, seconds, 10 seconds, 15 seconds or some other period of time), the system 100 can determine that a rescuer is not effectively or safely performing his rescue tasks, and in response, prompt one of the members of the intervention team to take corrective action and/or send an electronic command to the chest compression device 160 to begin automatically performing chest compressions on the patient and/or send an electronic command to the defibrillator 170 to begin automatically administering defibrillating shocks to the patient. However, if the score drops below the threshold level for less than the threshold length of time t, the system 100 can continue to monitor the user's performance, but not generate a prompt for corrective action or send commands to the chest compression device 160 or the defibrillator 170. Although example threshold scores and threshold lengths of time are described, these are merely illustrative examples. In practice, any values can be used. In some cases, the threshold score and the threshold length of time can be defined by a member of the intervention team and/or by a developer of the system 100, and/or defined based on one or more clinical protocols. Similarly, the system 100 can record the scores for the perfusion quality index over a period of time and generate prompts for corrective action when the rescuer is not effectively or safely performing his rescue tasks.

As described above, a user assigned to the recorder role (e.g., member 150d, also referred to as a “recorder”) can wear the wearable computing device 120b (corresponding to the recorder role) to assist him in with recording the tasks that were performed during the course of treating the patient. The recorder can be, for example, a user in charge of monitoring medication administration, the use of the defibrillator, and keeping an accurate time of the events.

In an example scenario, the leader has selected a protocol using the wearable computing device 120a, and the wearable computing devices 120a-d present information to the members of intervention team regarding tasks to be performed in accordance with the selected protocol. The wearable computing device 120b allows the recorder to record information regarding which tasks were performed by one of the team members, and at what time those tasks were performed to create an accurate record of the patient's treatment.

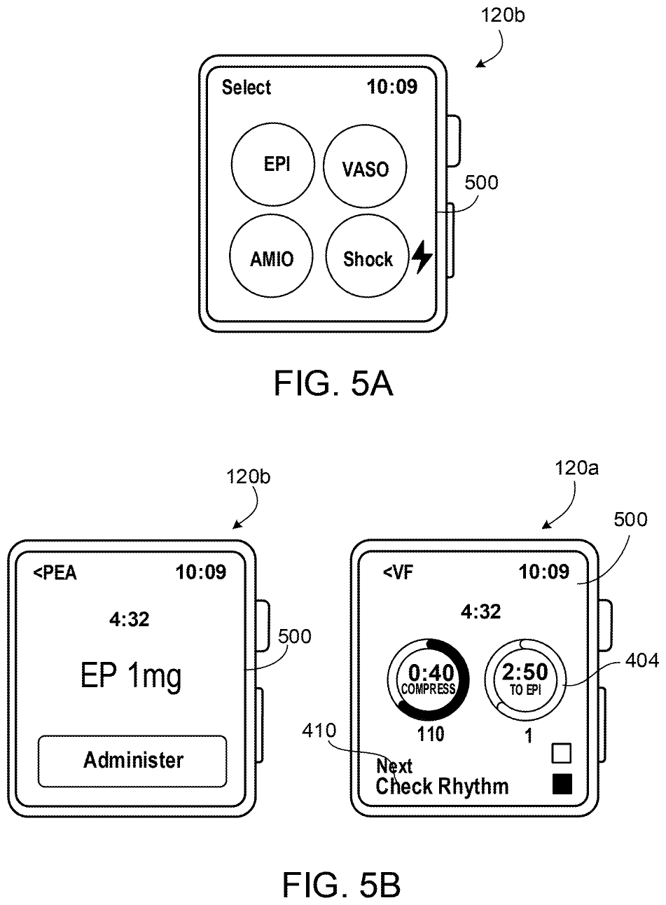

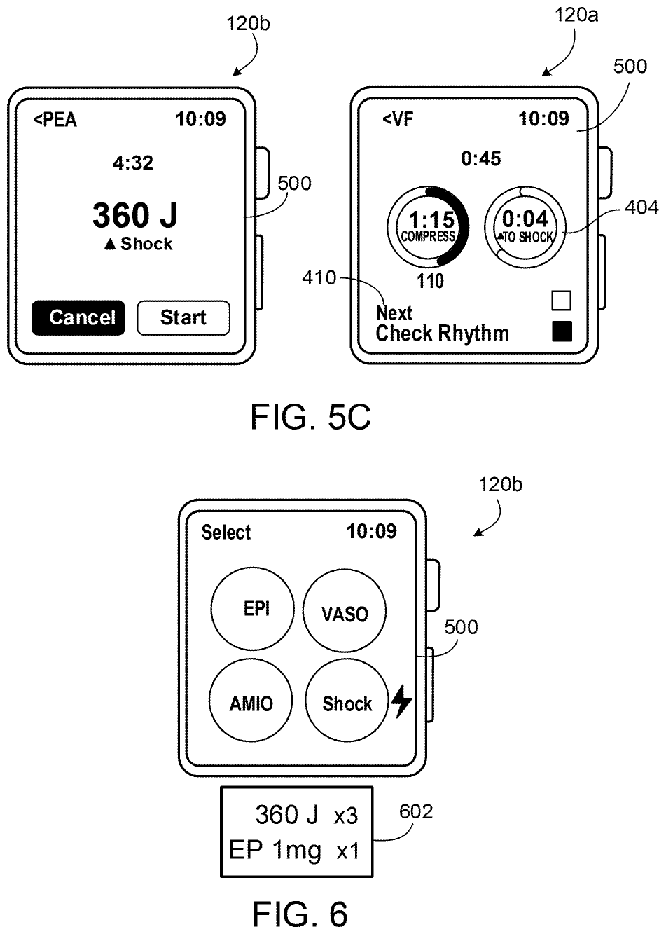

For example, as shown in FIG. 5A, the wearable computing device 120b can present a GUI 500 that displays a list of tasks that may be performed by one or more members of the intervention team. This list of tasks can be contextually filtered, such that the user is presented with only a subset of all possible tasks. For example, if the selected protocol specifies that a particular series of tasks be performed, the wearable computing device 120b can present only the most imminent tasks (e.g., the next N tasks) and/or the most recently scheduled tasks (e.g., the last M scheduled tasks), while not presenting other tasks. When the recorder determines that a particular task has been performed (e.g., by observing the performance of that task by one of the members of the intervention team or by observing the automatic performance of that task), the user selects that task from the GUI 500. For instance, the recorder may observe than epinephrine has been administered to the patient, and selects the “EPI” option on the GUI 500. Alternatively, or in addition, the recorder may observe that an intubation or placement of an intravenous line has been performed and selects the “intubation” or “IV line” option on the GUI 500. The recorder may also enter the time at which the task was performed.

As shown in FIG. 5B, in response, the GUI 500 requests that the recorder confirm the selection (e.g., by selecting the “administer” option). Upon confirmation, the wearable computing device 120b records the time at which the task was confirmed (e.g., by recording a timestamp), and transmits this information to the computing device 110 for storage. The wearable computing device 120b and/or the computing device 110 can also transmit this information to one or more of the other wearable computing devices 120a, 120c, and 120d. For instance, the wearable computing device 120b and/or the computing device 110 can transmit the time at which the administration of epinephrine was confirmed to the wearable computing device 120a. In response, the computing device 120a can update its GUI 400 to reflect the selection, for example by updating the upcoming task portion 404 of the GUI 400 to indicate when the next task (e.g., the task after the administration of epinephrine) should be performed. The computing device 120a can also update the task preview portion 410 of the GUI 400 to indicate the subsequent tasks that should be performed (e.g., tasks to be performed after the task shown in the upcoming task portion 404.

As another example, the recorder may observe than a defibrillation procedure has been performed on the patient, and selects the “Shock” option on the GUI 500. As shown in FIG. 5C, in response, the GUI 500 is updated to request that the recorder confirm the selection (e.g., by selecting the “Start” option). Upon confirmation, the wearable computing device 120b records the time at which the task was confirmed (e.g., by recording a timestamp), and transmits this information to the computing device 110 for storage. As above, the wearable computing device 120b and/or the computing device 110 can also transmit this information to one or more of the other wearable computing devices 120a, 120c, and 120d. For instance, the wearable computing device 120b and/or the computing device 110 can transmit the time at which the performance of defibrillation was confirmed to the wearable computing device 120a. In response, the computing device 120a can update its GUI 400 to reflect the selection, for example by updating the upcoming task portion 402 of the GUI 400 to indicate when the next task (i.e., the task after defibrillation) should be performed. The computing device 120a can also update the task preview portion 410 of the GUI 400 to indicate the subsequent tasks that should be performed (e.g., tasks to be performed after the task shown in the upcoming task portion 404. In certain cases, e.g., when defibrillation is automatically performed by the defibrillation device 270, the defibrillation device 170 may transmit a notification that a defibrillation shock has been applied, as well as the time at which defibrillation shock was applied, to any one or more of the wearable computing devices 120a-d.

In the above examples, a GUI presents information on a single screen, such that the user need not scroll through multiple screens of information. However, this need not be the case. In some implementations, a GUI can present information across several screens, and a user can scroll through each of the screens to access additional information. For example, as shown in FIG. 6, the GUI 500 of the wearable computing device 120b can display a list of tasks that may be performed by one or more members of the intervention team. However, the recorder can scroll downwards to access a lower portion 602. This lower portion 602 can present, for example, information regarding past defibrillation procedures that have been performed on the patient (e.g., the number of times that the patient had been previously defibrillated and the amount of energy that was applied), and information regarding past administrations of epinephrine (e.g., the number of times that epinephrine had been previously administered and the dosages). This can be useful, for example, as it allows the recorder to access recordation options corresponding to several different tasks, while also allowing him to access additional information regarding previously performed tasks (e.g., so that he can relay that information to others on the intervention team).

As described above, a user assigned to the rescuer role (i.e., the “rescuer”) can wear the wearable computing device 120c (corresponding to the rescuer role) to assist him in manually performing CPR on the patient.

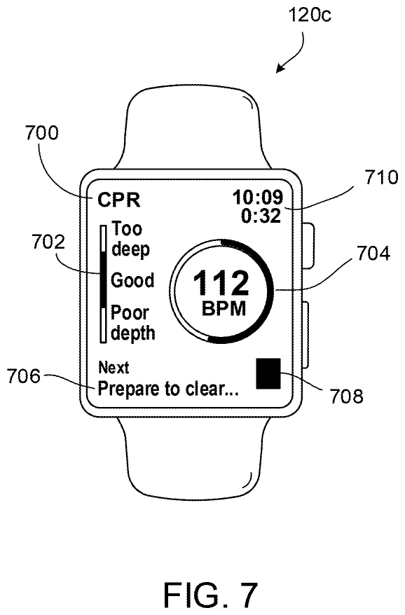

The wearable computing device 120c can obtain information regarding the effectiveness of the rescuer's efforts in performing CPR on the patient. As an example, referring to FIG. 7, the wearable computing device 120c can present a GUI 700 that presents information regarding the compression pressure of the rescuer's chest compressions, the depth of the compressions, and the compression rate of the rescuer's chest compressions. This allows the rescuer to determine whether he is applying the appropriate amount of pressure to the patient, whether his compressions are appropriately deep, and whether he is applying compressions at the correct rate.

The wearable computing device 120c can also instruct the user regarding the correct compression pressure, compression depth, and/or compression rate, and notify him if he is deviating from the correct procedure. For example, the GUI 700 can include a depth gauge 702 that indicates whether the rescue is achieving a proper compression depth (e.g., with a color-coded “good” indication), or whether he is achieving an improper compression depth (e.g., with color-coded “too deep” or “poor depth” indications). Although example labels are shown, it is understood that these are merely illustrative examples. Other labels can be used, depending on the implementation.

As another example, the GUI 700 can include a compression timer 704 that that indicates the period of time that the rescuer is tasked with performing chest compressions, the rate at which the rescuer is applying those compressions, and whether the rescuer is applying those compressions at the proper rate. For example, the compression timer 704 can include a numerical indicator that indicates the rate (e.g., compressions per minute) of the rescuer's compressions. The compression timer 704 can also include a ring representing the interval of time that the rescuer is tasked with performing chest compressions. For example, when the rescuer is initially tasked with performing chest compressions, the arc can be displayed as a full circle. As time passes, the arc gradually decreases in length. When the arc disappears, this indicates that the rescuer is to discontinue compressions. For example, a second rescuer can be notified to perform CPR. This is beneficial, as it allows the rescuer to quickly determine whether he is applying chest compressions for the appropriate amount of time, and whether he should discontinue chest compressions (and allow another member of the team to take over) to mitigate the effects of fatigue. The interval of time can vary, depending in on the implementation. For example, in some cases, the interval of time can be approximately 1 minute, 2 minutes, three minutes, or another interval of time.

In some cases, the arc can be color-coded to indicate the quality of the rescuer's efforts. For instance, in some cases, the arc can be color-coded to indicate whether the rate of compressions is too fast or too slow. For example, when the rescuer is performing chest compressions too quickly or slowly, the arc can be colored red. When the rescuer is performing chest compressions at the proper rate, the arc can be colored green.

In some cases, the pressure rate indicator 704 can also indicate whether the rate of compressions is relatively constant, or whether the rate of compressions has become inconsistent or uneven. In response, the pressure rate indicator 704 can indicate the uniformly of the chest compression (e.g., through the color coded arc). For example, the arc can be colored green if the chest compressions are being performed relatively uniformly, and red if the chest compressions are being performed relatively unevenly. This can be beneficial, for example, as it is often preferable for the rescuer to perform chest compressions at a uniform rate.

As with the wearable computing device 120a, the wearable computing device 120c can also provide the rescuer with information that summarizes the patient's condition, the task or tasks currently being performed, and future tasks or tasks to be performed. As an example, as shown in FIG. 7, the GUI 700 can include an upcoming task portion 706. The upcoming task portion 706 indicates an upcoming task that is to be performed in accordance with the selected protocol. The wearable computing device 120c also can include a treatment status portion 708. The treatment status portion 708 indicates the quality and/or efficacy of the intervention team's treatment of the patient. For example, the treatment status portion 708 can include an indication of a CPR quality index (e.g., a composite index based on factors such as compression rate, compression depth, compression pressure, pressure decay, and/or compression rate variability). This indications can be provided as a numerical score (e.g., on a quality scale of 1-10) and/or as a color score (e.g., on a quality scale of red, orange, and green, indicating poor quality, fair quality, and good quality, respectively). The GUI 700 can also include a time portion 710 and indicates the time that has elapsed since the leader initiated the intervention.

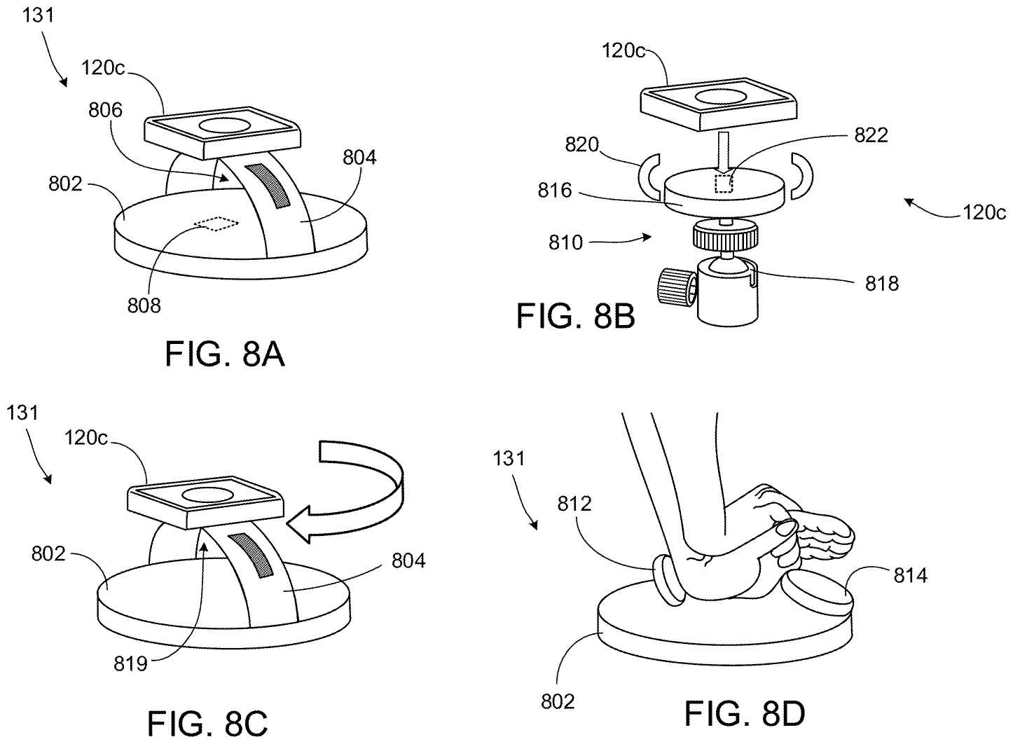

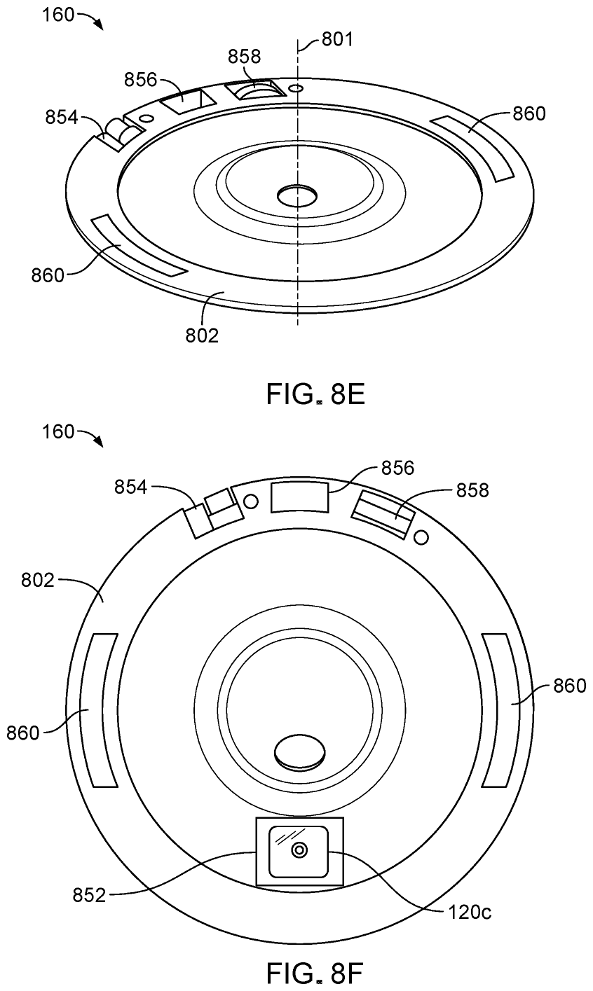

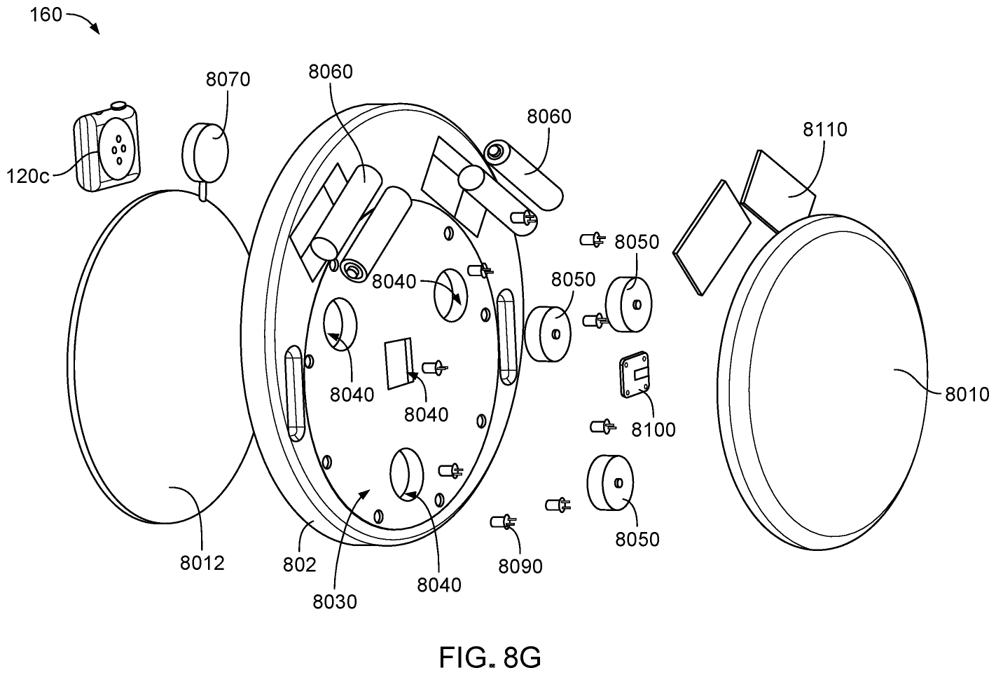

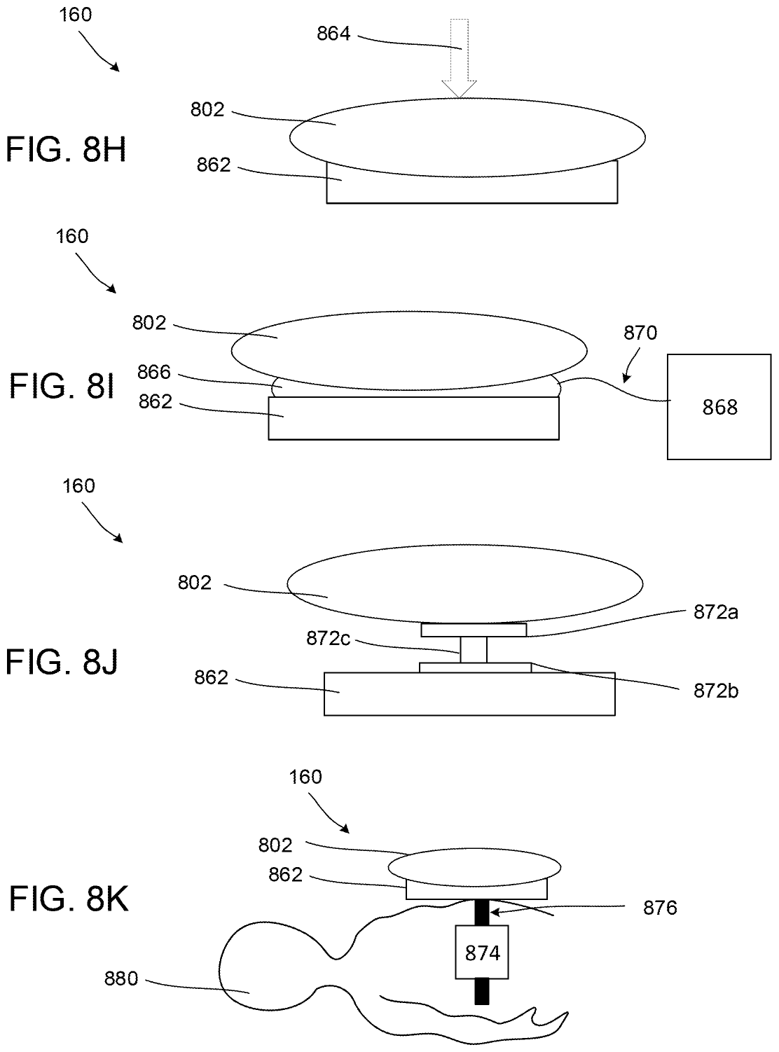

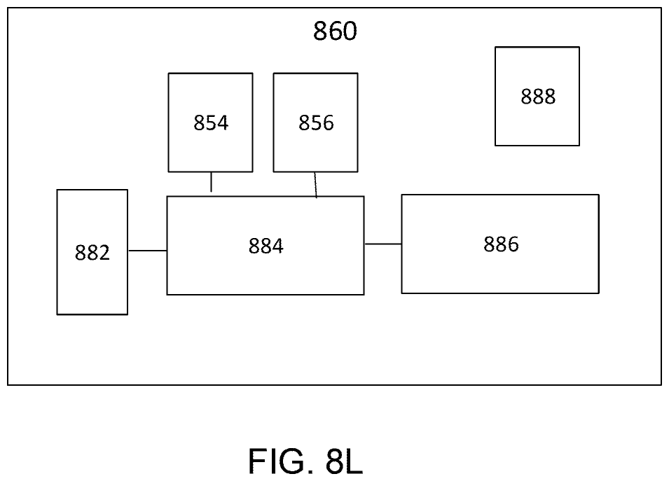

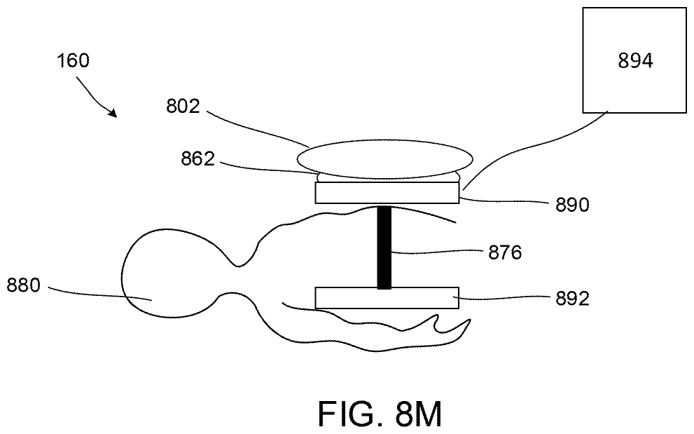

The wearable computing device 120c can obtain information regarding the effectiveness of the rescuer's efforts in performing CPR on the patient in various ways. For example, as shown in FIG. 8A, the wearable computing device 120c can be mounted to the chest compression device 160. In some implementations, the chest compression device is integrated with the defibrillator 170. In some implementations, the wearable computing device 120c is releasably secured to the chest compression device 160 and/or to the defibrillator 170. In some implementations, the computing device 120c may be permanently fixed to the chest compression device 120c and/or to the defibrillator 170, in which case the computing device 120c is not wearable.

As shown in FIG. 8A, the chest compression device 160 includes a compression plate 802 through straps 804. The straps 804 suspend the wearable computing device 120c above the compression plate 802, such that a space 806 is defined between the device 120c above the compression plate 802 and through which the user may place their hands.

The compression plate 802 can be constructed to include a cushion material (e.g., a soft plastic, compressible foam pad or silicone gel (for example, in a gel pack)) that comes into contact with the patient to reduce injury that may result from the rescuer's chest compressions. In some implementations, the cushion material of the compression plate 802 is affixed to a separate rigid support plate made of, e.g., plastic or metal, that does not deform when subjected to the forces typically applied to a patient during CPR. In these cases, the chest compression plate 802 does not conform as it might if it included a cushioned pad or gel pack alone. In some implementations, the rigid support plate includes an encasement or housing. In some implementations, the compression plate 802 does not include a rigid support plate and instead includes the cushion material alone. In some cases, the compression plate 802 can be constructed from a material that is resistant to solvents, such as those used to sanitize materials exposed to biological waste. This can be useful, for example, as it improves the durability of the compression plate 802 through multiple uses. The compression plate 802 can be relatively flat (e.g., a disc), or it can be shaped such that it conforms to the exterior surface of a human body. For example, in some implementations, the compression plate can include a rigid plastic material that is molded with a predefined shape or contour of a human body.

As explained above, in some implementations, the compression plate 802 includes an encasement or housing. The encasement or housing may be formed, e.g., in whole or in part from rigid plastic. The encasement or housing may include internal recesses or cavities in which one or more components of the chest compression device 160 can be fixed. Alternatively, or in addition, the encasement or housing may include recesses or openings formed on an external surface for receiving and fixing one or more components of the chest compression device 160.

For example, referring to FIG. 8A, the compression plate 802 can include one or more sensors 808, such as a pressure sensor, on an external surface or embedded within the compression plate 802. The pressure sensor 808 detects the amount of pressure that is applied to the compression plate 802, and transmits this information to a computing device, such as wearable computing device 120c (e.g., through a wired or wireless connection). For example, in some cases, the pressure sensor 808 can include a wireless transmitter (e.g., a Bluetooth radio) that allows the pressure sensor 808 is wirelessly transfer information to an electronic control module within the computing device 120c. The pressure information transmitted to the electronic control module can be used to signal to a user that too much pressure (e.g., a pressure outside of a predefined safe range) or too little pressure (e.g., a pressure outside of a range effective to lead to compression) is being applied. Example pressure sensors include, for example, pressure mapping sensors from Tekscan, Inc. (South Boston, Mass.), such as Tekscan Pressure Mapping Sensors and Tekscan Medical Sensors.

In another example, the sensors 808 on or embedded within the compression plate 802 includes an accelerometer. The accelerometer detects and outputs an acceleration signal indicative of the amount of acceleration to which the compression plate 802 is subjected. The accelerometer transmits the acceleration signal to a computing device, such as wearable computing device 120c (e.g., through a wired or wireless connection). For example, in some cases, the accelerometer can include a wireless transmitter (e.g., a Bluetooth radio) that allows the accelerometer to wirelessly transfer information to an electronic control module within the wearable computing device 120c. Alternatively, the accelerometer can transmit the acceleration signal to a processor within the compression device 160 (e.g., through a wired or wireless connection). The acceleration signal transmitted to the electronic control module can be used to signal to a user that the compression depths are too deep (e.g., a compression depth that is greater than a predefined effective range) or that the compression depths are not deep enough (e.g., a compression depth that less than a predefined effective range). The acceleration signal may be integrated by one or more of the wearable computing devices 120a-d or by the processor within the chest compression device 160 to obtain the compression depth. Example accelerometers include, for example, the MIS2DH 3-axis accelerometer available from STMicroelectronics.

In another example, the sensors 808 on or embedded within the compression plate 802 includes one or more gyroscopes. The gyroscopes detect and output an orientation signal indicative of the orientation of the compression plate 802 during compressions. The gyroscopes transmit the orientation signal to a computing device, such as wearable computing device 120c (e.g., through a wired or wireless connection). For example, in some cases, the gyroscopes can include a wireless transmitter (e.g., a Bluetooth radio) that allow the gyroscopes to wirelessly transfer information to an electronic control module within the wearable computing device 120c. Alternatively, the gyroscopes can transmit the orientation signal to a processor within the compression device 160 (e.g., through a wired or wireless connection). The orientation signal transmitted to the electronic control module can be used in combination with the acceleration signal from the accelerometer to provide more accurate depth information. Example gyroscopes include, for example, the L3GD20H 3-axis angular rate sensor available from STMicroelectronics.

In an example usage of the chest compression device 160 under manual operation, the rescuer places the compression plate 802 on top of the patient's chest, and places his hands in the space 806 (e.g., with interlocking fingers in CPR position). The rescuer then applies compressive pressure to the compression plate 802, thereby compressing the patient's chest in accordance with a CPR technique. The compression plate 802 can be beneficial, as it distributes the pressure applied by the rescuer, thereby reducing the likelihood of injury to the patient. As the rescuer performs compressions, the one or more sensors 808 measure information, such as the applied pressure, acceleration and angular rate, and transmit the measurements to the wearable computing device 120c. In turn, the wearable computing device 120c presents the information regarding the compressions to the rescuer (e.g., using the GUI 700). The compressions can be coupled with the manual or automatic application of one or more defibrillating shocks by the defibrillator in accordance with a CPR technique.

In some cases, the position of the wearable computing device 120c can be detachably secured to the chest compression device 160. In this way, the chest compression device 160 offers the convenience of allowing a user that is focused on the chest compression device 160, as compressions are performed, to also view in the same general area relevant information output from the computing device 120a. Alternatively, the user can decide to detach the computing device 120a and wear it instead (e.g., on the user's wrist) during application of the compressions. In some implementations, a position and/or orientation of the wearable computing device 120c can be adjusted with respect to the compression plate 802 of the chest compression device 160. For example, as shown in FIG. 8B, the wearable computing device 120c can be mounted onto a swivel mechanism 810. The swivel mechanism 810 includes a mounting plate 816 positioned atop a ball and socket joint 818. The wearable computing device 120c is detachably secured to the mounting plate 816 by, e.g., one or more clips 820 and/or magnets 822. The swivel mechanism 810 may in turn mounted onto the strap 804. Thus, the wearable computing device 120c can be rotated with respect to the compression plate 802, such that the rescuer (or another user) can more readily view the wearable computing device 120c during the course of treatment, without otherwise adjusting the position of the compression plate 802.

In some cases, the compression plate 802 can include grips or supports that physically guide the user in performing proper chest compressions. For example, as shown in FIG. 8D, the compression plate 802 (for simplicity, shown without the wearable computing device 120c and straps 804) can include a raised heel 812 to allow the rescuer to rest the heel of the hand against it and position the palm of the hand in proper CPR position. The compression plate 802 can also include a grip 814 at the front of the compression plate 802 to prevent slippage of the rescuer's hand. The grip 814 can also include grooves, such that the rescuer can position the fingers in an ergonomic position, thereby reducing fatigue.

Other configurations of the chest compression device 131 are also possible. For example, FIG. 8E is a schematic that illustrates a perspective view of an example of a chest compression device 160. FIG. 8F is a schematic that illustrates a top view of the exemplary chest compression device 160 shown in FIG. 8E. FIG. 8G is a schematic that illustrates an exploded view of an exemplary chest compression device 160.

As in the configuration shown in FIG. 8A, the chest compression device 160 shown in FIG. 8E includes a compression plate 802. In some implementations, the compression plate 802 has an approximately circular shape so that it may be shared between rescuers potentially located opposite each other during use of the device 160. Additionally, by providing an approximately circular shape, the chest compression device 160 can be quickly placed on a patient's chest during use without concern as to whether the device 160 has been properly oriented. That is, the device 160 may be rotated to any position about a central axis 801 extending through a plane of the device 160 when applied to a patient's chest.

As explained herein, the device 160 may be configured to allow a wearable computing device, such as any of wearable computing devices 120a-120d, to be releasably attached to a surface of the chest compression device 160. For example, referring to FIG. 8F, the chest compression device 160 may include a recess 852 in which the computing device 120c may sit. The computing device 120c may be releasably secured within the recess 852 through friction, clips, magnets, although other attachment configurations are also possible. The recess 852 may be located on a top surface of the compression plate 802 such that when the computing device 120c is attached, the face of the device 120c is visible to the team member operating the chest compression device 160 or to other team members in the vicinity of the device.

As explained above, the chest compression devices described herein may include one or more sensors 808, such as a pressure sensor to detect the amount of pressure applied to a patient during operation of the chest compression device, an accelerometer, and/or a gyroscope. In addition or as an alternative, the chest compression devices may include one or more other sensors. The one or more other sensors may be used to record patient data or other information related to the operation of the chest compression device and/or the defibrillator. For example, in some implementations, the one or more other sensors may include, but are not limited to, proximity sensors to measure the absolute or relative displacement of the chest compression device (and thus the distance that the patient's chest is compressed), an electrocardiogram (ECG) rhythm monitor to record electrical and muscular functions of a patient's heart, a tissue perfusion sensor to monitor tissue perfusion in the patient, an oxygen perfusion sensor to monitor an amount of oxygen perfusion of the patient, a capnometer to record concentration or partial pressure of CO₂ in a patient's exhaled air, and a brain oxygenation sensor to measure an amount of brain oxygenation of the patient, a blood pressure sensor to measure a blood pressure of the patient. The plate or pad 802 of the chest compression device 160 may include one or more recesses, such as recesses 854, 856, for receiving the one or more sensors. As with the computing device 120c, the one or more sensors may be releasably secured (e.g., through friction with the recess side walls, clips, and/or magnets, among other securing configurations) within the recesses 854, 856 of the chest compression device 160. In some implementations, the sensor portion of the one or more sensors may actually be placed on the body of the patient (e.g., the capnometer may be placed in a patient's airway to measure CO₂, a heart rate sensor may be placed on the patient's neck or limb, an ECG monitor may be placed on a patient's chest) and the electronic processors and/or memory for receiving the data measured by the sensor is located within the one or more recesses 854, 856 of the chest compression device 160. In some implementations, the electronic processors and/or memory that receives the measurement data from the one or more sensors located on the patient's body are communicatively coupled to the one or more sensors through a wire or wirelessly. In some implementations, the one or more sensors wireless communicate (e.g., transmit sensor measurements) directly with the computing device 120c that is worn by a team member or secured to the chest compression device 160. The wireless communication may employ, e.g., Bluetooth protocol or a local wireless area network.

In some implementations, the chest compression device 160 includes one or more light emitting elements 858, such as a light emitting diode. In some implementations, the chest compression device includes multiple light emitting elements, in which each light emitting element emits a different color wavelength of light representative of a chest compression quality. For example, in some cases, the chest compression device 160 includes a green LED that lights up when the chest compressions being performed are within predetermined acceptable parameters. The chest compression device 160 may also include a red LED that lights up when the chest compressions being performed deviate from predetermined acceptable parameters, prompting a team member to make adjustments to the chest compression procedure. Other colors and indicators of chest compression quality are applicable as well.

In some implementations, the light emitting element 858 is embedded within the compression plate 802 of the chest compression device 160, and the compression plate 802 is formed from a material that is transparent or semi-transparent to the wavelength of light emitted by the light emitting element 858.

In some implementations, the chest compression device 160 includes an internal power source, e.g., a battery, for powering the one or more sensors, the light emitting element 858, and/or the wearable computing device 120c when the wearable computing device is secured to the chest compression device 160.

As explained herein, in some implementations, the chest compression device 160 can be configured to operate in automatic mode, rather than manual mode in which a user applies the force to generate the compressions. In the automatic mode, the chest compression device 160 may be secured to a patient using a strap (see, e.g., strap 876 in FIG. 8K) that also couples to the chest compression device 160. In some implementations, the chest compression device 160 includes openings or slots 860 through which ends of the strap may be secured to the chest compression device 160. The strap length may be adjustable so that it can fit around different sized patients. For instance, the strap may have hook and loop fasteners on its ends that allow the strap to be fastened to itself at different positions, thus allowing the strap to form different sized loops when secured around the patient's chest.

FIG. 8G is a schematic that illustrates an exploded view of another example of the chest compression device 160. As shown in FIG. 8G, the chest compression device 160 includes a chest compression plate 802. The chest compression plate 802 may be formed, e.g., of a rigid plastic. The chest compression plate 802 includes a first recess 8030 on a first side of the plate 802. The first recess 8030 is sized to receive a first defibrillator pad 8010 of a defibrillator. The first defibrillator pad 8010 may be used to provide defibrillation shocks to a patient when the device 160 is secured or placed on the patient. During operation of the chest compression device 160, the first defibrillator pad 8010 is coupled to a power source.

A second recess may be formed on a second opposite side of the chest compression plate 802 and sized to receive a second pad 8012. The second pad 8012 can include, e.g., a foam cushion to reduce discomfort on a user's hand during manual operation of the chest compression plate 802.

Both the pads 8010, 8012 may be secured to the chest compression plate 802 using, e.g., an adhesive glue. In some implementations, the adhesive glue used for one or both of the pads 8010, 8012 is a temporary adhesive that allows the pads to be removed and replaced after use. In some implementations, the defibrillator pad 8010 includes a self-adhesive defibrillator pad, similar to the HeartStart M3713A MRX defibrillation pad from Koninklijke Philips N.V.

Within the first recess 8030, one or more third recesses 8040 may be formed in the chest compression plate 802. The one or more third recesses 8040 can be sized to receive one or more sensor components 8050 and/or a microprocessor 8100. The one or more sensor components 8050 and/or the microprocessor 8100 can be secured within the corresponding third recesses 8040 through friction with the sides of the third recesses 8040, with clips, and/or with cover plates that are secured to the chest compression plate 802, among other securing mechanisms. The one or more sensor components 8050 can include, as described herein, sensors such as pressure sensors, accelerometers, angular rate sensors, among other types of sensors. The microprocessor 8100 can be electronically coupled to the one or more sensors 8050 wirelessly or through wiring embedded in the chest compression plate 802. The microprocessor 8100 can include special purpose circuitry such as an ASIC chip. In some implementations, the microprocessor includes memory on which computer program instructions are stored, in which during operation of the chest compression device 160, the microprocessor 8100 executes the stored instructions. For instance, in some implementations, the microprocessor 8100 executes instructions for receiving sensor data from the one or more sensors 8050, processing the sensor data (e.g., converting accelerometer signals into position information), and transmitting the processed sensor data to one or more of the wearable computing devices 120a-d.

The chest compression plate 802 further includes a power source 8060 for powering the one or more sensors 8050 and the microprocessor 8100, among other components of the chest compression device 160. The power source 8060 may include, e.g., batteries such as NiCd or lithium ion rechargeable batteries. In some implementations, the power source 8060 provides power to the defibrillation pads. The power source may include power sources having sufficient power to generate a predefined number of defibrillations including, e.g., between 50 and 200 defibrillations having a potential applicable for a patient suffering from heart failure. The total number of defibrillations may depend, e.g., on the potential applied during each defibrillation and whether the defibrillation is operated in a pacer mode or not. In an example, the power source may be operable to provide 260 defibrillation discharges each at a level of approximately 200 Joules corresponding to approximately 576 minutes of operating time. The batteries may be secured within a recess within the chest compression plate 802. The power source may be secured using friction, with clips, and/or with cover plates (such as cover plates 8110).

In some implementations, the chest compression plate 802 includes one or more light emitting elements 8090, such as light emitting diodes. The light emitting elements 8090 may be fixed to the first side and/or to the second side of the chest compression plate 802. As described herein, the light emitting elements 8090 can emit a different colors of light representative of a chest compression quality.

As also shown in FIG. 8G, a wearable computing device, such as wearable computing device 120c, may be fixed to the second side of the chest compression plate 802. In some implementations, the chest compression device 160 also includes a charger component 8070, such as a magnetic charger. The charger component 8070 can be used or wirelessly re-charging the power source of the chest compression device 160. The charging component 8070 can be electrically coupled to the power source through wiring within the compression plate 802. In some implementations, the charger component 8070 is for coupling to an external power source that provides power to the first defibrillator pad 8010.

FIGS. 8H-8K are schematics that illustrate different exemplary configurations of the chest compression device 160. In FIG. 8H, the chest compression device 160 may be operated in manual mode, where pressure is applied from a team member pressing their hands down on the chest compression device 160 when the device 160 is positioned on the patient's chest. As described herein, the chest compression device 160 may include a compression plate 802 coupled to a cushion layer 862 formed of a cushion layer 862 (e.g., a foam or gel) so that the force 864 applied to the device 160 may be distributed uniformly and without causing pain to the patient.

In FIGS. 8I-8K, the chest compression device 160 may be configured to operate in an automatic mode, in which the compression force is generated using mechanical devices rather than a team member pressing down on the device 160. For instance, in FIG. 8I, the chest compression device includes an inflatable bladder 866 located between the compression plate 802 and the cushion 862. The bladder 866 may be formed from rubber or other expandable material and may be coupled to a pneumatic tube 870 which, in turn, may be coupled to a compressor device 868 that supplies air to or withdraws air from the bladder 866. During operation of the chest compression device 160, the device 160 may be secured to a patient using, e.g., a strap that holds the device 160 in place against the patient's chest. The compressor 868 then may be activated to fill the bladder with air causing a downward force to be generated against the patient's chest, in a manner similar to a manually initiated chest compression. At the end of the compression, the compressor 868 may then withdraw air from the bladder 866, such that the force applied to the patient's chest decreases. The compressor 868 may include a wireless transceiver to receive electronic commands from, e.g., one of the wearable electronic devices 120a-120d, in which the electronic commands may direct the compressor to begin inflating or deflating the bladder 866. The electronic commands may be communicated through a network protocol such as, e.g., Bluetooth or through a wireless local area network. In some implementations, the compressor may be integrated into the chest compression device and coupled directly to the bladder 866 such that a pneumatic tube is not needed.

In some implementations, the chest compression device 160 includes a mechanical piston that can be used to generate the compression force. For example, in FIG. 8J, the chest compression device 160 includes a mechanical piston 872 located between the compression plate 802 and the cushion layer 862. The piston may include an upper plate 872a coupled to the compression plate 802 and a lower plate 872b coupled to the cushion layer 862, in which one or more moveable center pistons 872c are arranged between each plate 872a, 872b. The center piston 872c may include, e.g., a pneumatic piston. During operation of the chest compression device 160, the device 160 may be secured to a patient using, e.g., a strap that holds the device 160 in place against the patient's chest. The mechanical piston 872 may include a wireless transceiver to receive electronic commands from, e.g., one of the wearable electronic devices 120a-120d, in which the electronic commands may direct the piston to begin increasing or decreasing the distance between the upper plate 872a and lower plate 872b. Because the device 160 is secured to the patient, the increase in separation distance gives rise to a force against the patient's chest in a manner similar to a manual chest compression. The electronic commands may be communicated through a network protocol such as, e.g., Bluetooth or through a wireless local area network.

In some implementations, the chest compression device 160 is coupled to a strap tensioner device 874. As explained herein, the chest compression device may be secured to a patient 880 using a strap 876 that wraps around the patient's chest and back such that the chest compression device 160 is held in place on the patient's chest. The strap 876 may be secured to the chest compression device 160 through one or more slots in the housing of the device 160 that are wide and thick enough to receive the strap 876. In the example shown in FIG. 8K, the strap 876 also is coupled to a strap tensioner 874 that is capable of increasing or decreasing the tension in the strap 876 to increase or decrease, respectively, the compression felt by the patient during operation of the chest compression device. For instance, in some cases, the strap tensioner 874 may receive both a first end and a second end of the strap 876 such that the strap 876 is arranged in a loop. The loop can be positioned around the patient's chest. During operation of the chest compression device 160, the strap tensioner 874 may decrease the length of the strap forming the loop to increase a tension around the patient's chest or may increase a length of the strap to decrease a tension around the patient's chest. The strap tensioner 874 may include a wireless transceiver to receive electronic commands from, e.g., one of the wearable electronic devices 120a-120d, in which the electronic commands may direct the tensioner 874 to begin increasing or decreasing the tension of the strap 876. The electronic commands may be communicated through a network protocol such as, e.g., Bluetooth or through a wireless local area network.

In some implementations, an advantage of the automatic chest compression device is that it may be used to perform chest compressions without a user in close proximity to the patient. Accordingly, when a defibrillation shock is applied, there is no need for team members to clear away from the patient.

As explained herein, the chest compression device 160 may include a position sensor or accelerometer to determine the depth of a compression when applied to a patient. When force is applied in a downward manner against the patient's chest, however, this may cause a mattress underlying the patient also to compress, leading to an inaccurate measurement of compression depth. To compensate for the movement of an underlying mattress, the chest compression device may include a second accelerometer underneath the patient's back. The acceleration information from the second accelerometer may be used to provide a correction factor for position information calculated from the first accelerometer. For example, the accelerometer signal from the first accelerometer may be integrated (e.g., by the microprocessor within the chest compression device 160 or within one of the wearable computing devices 120a-d) to provide a first compression depth. The accelerometer signal from the second accelerometer may be integrated to provide a second compression depth. This second compression depth represents the movement of the entire patient's body in response to the application of force, rather than just movement of the patient's chest. The second compression depth then may be subtracted (in the chest compression device microprocessor or in one of the computing devices 120a-d) from the first compression depth to provide a corrected and more accurate overall compression depth of the patient's chest. In some implementations, the second accelerometer may be coupled to or embedded along the strap (e.g., see strap 876 in FIG. 8K) such that when the strap is secured to the patient, the location of the second accelerometer is positioned on the patient's back. In some implementations, the second accelerometer may be secured to or embedded in the mattress itself. The second accelerometer may include a wireless transceiver to send the acceleration data back to the microprocessor of the chest compression device 802 or to one of the wearable computing devices 120a-d. Alternatively, the second accelerometer may be coupled to the microprocessor through a wired connection. The wires of the wired connection may extend within or along an outer surface of the strap that couples the chest compression device to the patient.

Though the chest compression device has been described herein as being operable in either manual or automatic mode, the chest compression device may be configured so that it can be switched between manual or automatic operation. For example, in any of the configurations described herein, the strap coupled to the chest compression device may be detached such that a team member can position the chest compression device as needed for manual operation. In some implementations, the compression bladder 866 may be removably attached to the compression plate 802 and/or to the cushion layer 862. For example, the compression bladder 866 may be attached to the compression plate 802 and/or to the cushion layer 862 through a hook and loop fastener such that it can be easily removed from the compression plate 802 and from the cushion layer 862. In some cases, when the compression bladder 866 is removed from the configuration shown in FIG. 8I, the cushion layer then can be attached directly to the compression plate 802 through the hook and loop fastener as shown in FIG. 8H.

In some implementations, the chest compression device is configured to aggregate measurement data from the one or more sensors, process the data to obtain information relevant to the one or more team members involved in the CPR procedure, and to output the information either to a display and/or to one or more of the wearable computing devices 120a-120d. For example, FIG. 8L is a schematic that illustrates an example of the electronic components that can be included in a chest compression device 160. The chest compression device 160 is represented as a block and includes a transceiver 882 for receiving and transmitting electronic signals, such as electronic command signals for automatically operating the chest compression device or data measurements obtained from the one or more sensors. The chest compression device 160 also may include one or more electronic processors 884 that are coupled to one or more patient sensors 890, 892 (e.g., capnometer, blood pressure sensor, accelerometer, displacement sensor, compression device pressure sensor, tissue and oxygen perfusion sensor, brain oxygenation sensor, among others) and that can be configured to process data received from the one or more sensors, save data to an associated memory 886, and transmit data to the transceiver 884. The chest compression device 160 also may include a power source 888 that powers the one or more sensors, the transceiver 882, the electronic processor 884 and the memory 886.

As an example of the data processing that may be performed by the processor 884, in some implementations, the processor 884 may take as an input the chest compression device acceleration rate from an accelerometer sensor, the chest compression pressure from the pressure sensor, and generate a value indicative of a compression quality index. The compression quality index may be output by the device 160 to one or more of the wearable computing devices 120a-120d and/or to a display to serve as a monitoring index to inform the code leader and rescuer about the quality of CPR. In another example, the processor 884 may take as an input CO₂ levels from the capnometer and brain oxygenation levels from a brain oxygenation sensor to generate a composite patient perfusion index. Again, the composite perfusion index may be output by the device 160 to one or more of the wearable computing devices 120a-120d and/or to a display so that additional information is available to team members in case adjustments need to be made to the resuscitation process. In another example, the chest compression device 160 may receive, at the transceiver 882, one or more commands from a wearable computing device 120a-120d. The command may be transmitted to the processor 884, which, in turn may process the command. For instance, the command may include instructions to perform automatic chest compressions. Depending on the configuration of the chest compression device and the data received from the wearable computing device 120a-120, the processor may instruct, e.g., the compressor 868 to inflate and deflate the compression bladder 866 according to a predetermined cycle and a particular pressure so as to achieve a desired compression. In some other implementations, the processor 884 may issue instruct, e.g., the piston 872 to increase and decrease the displacement between plates 872a, 872b according to a predetermined cycle and separation distance so as to achieve a desired compression. In some other implementations, the processor 884 may issue instruct, e.g., the strap tensioner 874 to increase and decrease the tension in strap 872 according to a predetermined cycle so as to achieve a desired compression.

As explained herein, in some implementations, one or more of the wearable computing devices 120a-120d may issue commands to a defibrillator 170 for automatically applying a defibrillation shock to the patient during the CPR process as necessary. In some implementations, the defibrillator 170 can be integrated as a component part of the chest compression device. For instance, as shown in FIG. 8G, the chest compression device 160 includes a first defibrillation pad 8010 attached to a first surface of the chest compression plate 802. FIG. 8M is a schematic that illustrates another example of a chest compression device 160 that includes a defibrillator 170. As in other examples described herein, the chest compression device 160 may include a compression plate 802 as well as a cushion layer 862 to reduce injury to the patient during compressions.

The defibrillator 170 may be formed of a a first defibrillator pad 890 and a second defibrillator pad 892. The first defibrillator pad 890 may be attached to a bottom surface of the cushion layer 862. Alternatively, in other configurations, the first defibrillator pad 890 may be attached to a bottom surface of the compression plate 802 in place of the cushion layer 862. Alternatively, in other configurations, the, e.g., the first defibrillator pad 890 may be attached to a bottom surface of the compression bladder 866 (see FIG. 8H) in place of the cushion layer 862. The second defibrillator pad 892 may be located beneath the patient 880, e.g., under the patient's back. The second pad 892 may be coupled to the chest compression device 160 using the strap 876. The strap 876 also may serve to hold the second defibrillator pad 892 and the chest compression device 160 in place in the proper locations for chest compression and defibrillation. As described herein, the strap 876 may be adjustable so that the chest compression device and pads 890, 892 can be fitted around different sized patients. In some implementations, the defibrillator pads 890, 892 include self-adhesive defibrillator pads.

In some implementations, the defibrillator pad 890 are removably secured to the compression bladder 866, to the cushion layer 862, and/or to the 802 through, e.g., an adhesive glue or hook and loop fasteners. In some implementations, sides of the defibrillator pads 890, 892 that contact the patient include a skin-friendly medical adhesive such as, e.g., 1509 Transparent Polyethylene Double Sided Medical Tape from 3M®. Alternatively, the medical adhesive can include a conductive adhesive that aids in transferring the electrical signal from the defibrillator pads

In some implementations, the first and second defibrillator pads 890, 892 are coupled to a power source 894 that provides the energy for charging the pads 890, 892 during a defibrillation. The power source may be internal to the chest compression device 160 (e.g., see power supply 8060 in FIG. 8G) or external to the chest compression device 160. In some implementations, the processor 884 of the chest compression device may receive commands from one of the wearable computing devices 120a-120d to perform a defibrillation. The command may include information as to the power (e.g., joules), voltage, charge duration, time between defibrillation charges (shocks), number of defibrillation charges to be applied, and/or whether the defibrillation charge setting is synchronized/non-synchronized. Upon receiving the command, the processor may direct the power supply 894 to apply the specified defibrillation charge to one or both of the first and second defibrillation pads 890, 892.

In some implementations, the processor 884 of the chest compression device 160 may instruct the defibrillator 170 of the device 160 to perform defibrillations in a manual mode (e.g., where shocks are not applied until a trigger signal is received from a user operating the chest compression device 160 and/or one of the wearable computing devices 120a-d), in an automatic rescue mode (e.g., where shocks are applied for a patient suffering from heart failure), or in an automatic pacer mode (e.g., where microshocks are applied to maintain a predefined heart rate). The instruction to operate in a manual, automatic rescue mode, or automatic pacer mode may be sent by the processor 884 in response to a user command (e.g., from one of the wearable computing devices 120a-d or from a switch on the chest compression device 160).

In some implementations, the automatic modes of the defibrillator 170 (e.g., the automatic rescue mode or the automatic pacer mode) apply defibrillation shocks in response to a detected condition of the patient. For example, in some cases, the one or more sensors of the chest compression device 160 may include a heart rate sensor. Alternatively, a heart rate sensor may be separate from the chest compression device 160 but coupled to the device 160 to provide heart rate information to the processor 884 and/or to a wearable computing device 120a-d. Based on the received heart rate information, the processor 884 of the chest compression device 120a-d or the wearable computing device 120a-d can determine whether a shock is necessary and when a shock is necessary, cause the defibrillator 170 to apply the shock to the patient through the defibrillation pads. For example, in the automatic pacer mode, the processor 884 and/or the wearable computing device 120a-d may observe the heart rate and determine that the rate is within a predefined range that is below a desired heart rate (e.g., if the detected heart rate is below 65 beats per minute (bpm) but above 5 bpm, below 60 bpm but above 5 bpm, below 55 bpm but above 5 bpm, below 50 bpm but above 5 bpm, or below 45 bpm but above 5 bpm, among others). In response, the defibrillator 170 may be instructed by the processor 884 and/or one of the computing devices 120a-d to apply one or more microshocks to induce the heart rate to increase to a desired value or range (e.g., between 70 bpm and 150 bpm). In automatic recovery mode, the processor 884 and/or the wearable computing device 120a-d may observe that the rate is too low or non-measurable. For example, it may be determined that the heart rate is 10 bpm or less. In response, the defibrillator 170 may be automatically instructed by the processor 884 and/or one of the computing devices 120a-d to apply one or more shocks to the patient to recover the heart beat.

In some implementations, such as when the defibrillator 17 is in manual mode, the processor 884 and/or one of the wearable computing devices 120a-d may output a recommendation to apply a shock in response to determining that the patient's heart rate is below a predefined range, such as any of the ranges described above. In some cases, the recommendation may include a suggestion of a potential to apply during defibrillation to recover a specified heart rate. For example, in some implementations, one of the wearable computing devices 120a-d or the processor 884 is configured to determine, from the patient information obtained from the one or more sensors coupled to the patient and/or within the chest compression device 160, a patient state indicative of cardiac dysrhythmia. In response to this determination, the one or more wearable computing devices 120a-d and/or the processor 884 outputs to a display (such as the display of the one or more wearable computing devices 120a-d) a recommendation to activate or deactivate the defibrillator

In some implementations, the one or more computing devices 120a-d may output a warning that a shock is about to be applied. For example, the warning can be output to the displays of the wearable computing devices 120a-d. The warning can include illuminating one or more light emitting elements on the chest compression device 160.

As described above, a user assigned to the respiration monitoring role (i.e., the “respiration monitor”) can wear the wearable computing device 120d (corresponding to the respiration monitoring role) to assist him in monitoring the respiration of the patient during the course of treatment.

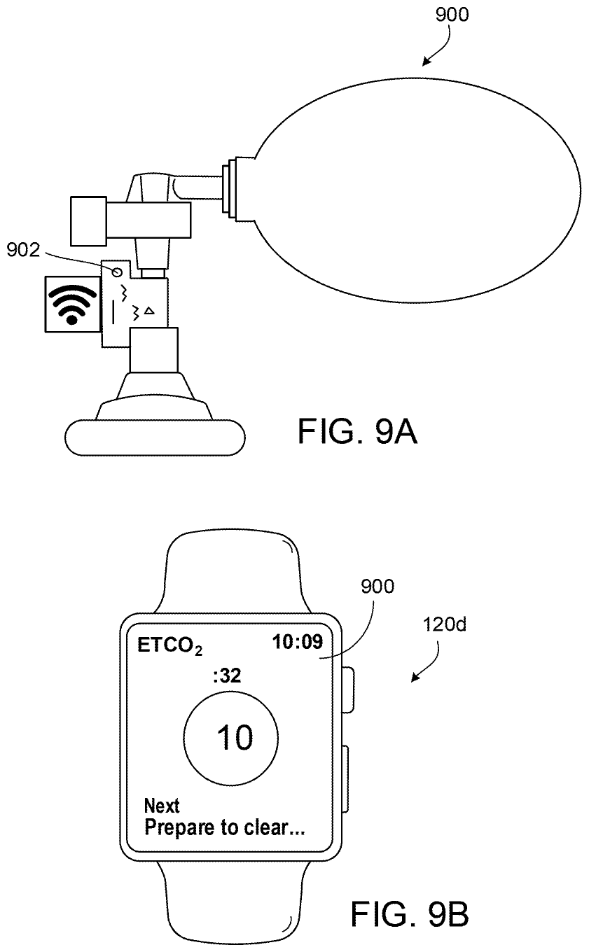

For example, if the patient has adequate blood flow to his lungs, the production of carbon dioxide by the patient's tissues will be appropriately eliminated by the lungs. Hence, elevated levels of end tidal CO₂ (ETCO₂) can be a reflection of appropriate resuscitation efforts. Referring to FIG. 9A, the wearable computing device 120d can obtain information regarding the ETCO₂ of the patient through a capnometer 902 (a sensor for measuring the concentration or partial pressure of CO₂) positioned along the airway of a bag valve mask 900. As the respiration monitor operates the bag valve mask 900 to manually provide positive pressure ventilation to the patient, the capnometer 902 obtains sensor measurements regarding the concentration of partial pressure of CO₂ of air exhaled from the patient's lungs. In some cases, the capnometer 902 may be communicatively coupled to the wearable computing device 120d (e.g., through a wired or wireless connection), such that the respiration monitor can observe the ETCO₂ of the patient. For example, as shown in FIG. 9B, the wearable computing device 120d can present a GUI 900 that displays the ETCO₂, along with other information regarding the patient (e.g., the time that was elapsed since the leader initiated intervention). In some cases, the capnometer 902 can be positioned along the airway of an endrotracheal tube to measure the ETCO₂ of the patient, for example when the patient is intubated. For the patient's ETCO₂ is low (e.g., below a particular threshold value, such as 10), the wearable computing device 120d can prompt the user to adjust the respiratory resuscitation. The wearable computing device 120d records and transmits this information to the computing device 110 for storage. The wearable computing device 120d and/or the computing device 110 can also transmit this information to one or more of the other wearable computing devices 120a-c. For example, the wearable computing device 120d and/or the computing device 110 can transmit information indicating that the patient's ETCO₂ is low to the wearable computing device 120c, prompting the rescuer to adjust his compression efforts. Alternately or in addition, if the intervention team is treating the patient manually and the patient's ETCO₂ is low, the wearable computing device 120d can send a command to the chest compression device 131 to begin performing chest compressions on the patient and/or a command to the defibrillator 132 to begin administering defibrillating shocks to the patient.

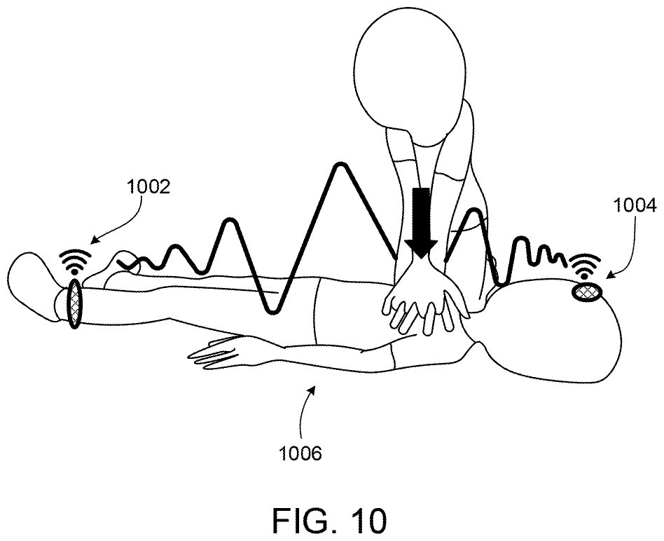

In some implementations, the electronic system 100 can also include one or more additional sensors for measuring other aspects of a patient's condition. For example, referring to FIG. 10, the electronic system 100 can further include sensors 1002 and 1004 that measure a patient's perfusion. The sensors 1002 and 1004 are placed on the patient's body 1006 (e.g., on the extremities of the patient, such as his arms or legs, or his forehead), and measure properties such as the patient's vascular flow or tissue oxygenation. Example sensor include laser Doppler flow sensors (e.g., scanning laser Doppler flowmetry sensors), spectroscopy (NAIR) sensors, and tissue saturation monitors.

Information gathered by the sensors 1002 and 1004 can be transmitted to other components of the electronic system 100 (e.g., the computing system 110 and/or one or more wearable computing devices 120a-d through a wireless network connection) to provide the members of the intervention team with information regarding the patient's condition. For example, in some cases, the information gathered by the sensors 1002 and 1004 can be used to determine the CPR quality index and/or a perfusion quality index. As another example, in some cases, the electronic system 100 can determined, based on information gathered by the sensors 1002 and 1004, that the patient's perfusion is poor. In response, the electronic system 100 can notify one or more of the members of the intervention team (e.g., by presenting an alert or notification using one or more of the wearable computing devices 102a-d) to modify or otherwise improve the resuscitation efforts. Alternately or in addition, if the intervention team is treating the patient manually, the electronic system 100 can send a command to the chest compression device 131 to begin performing chest compressions on the patient and/or a command to the defibrillator 132 to begin administering defibrillating shocks to the patient.

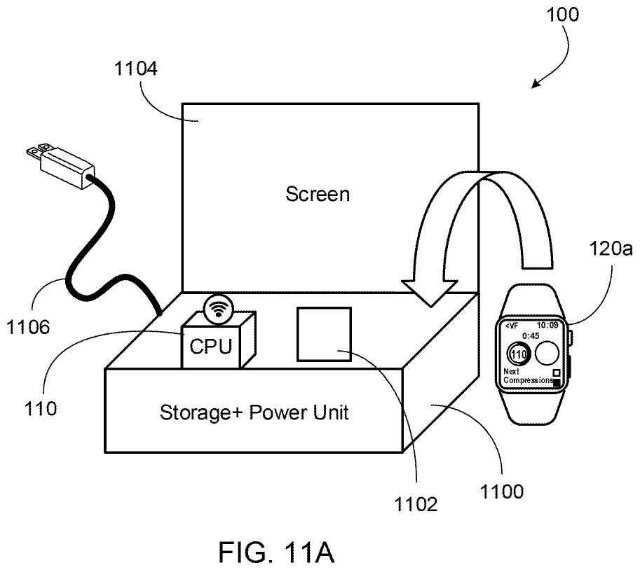

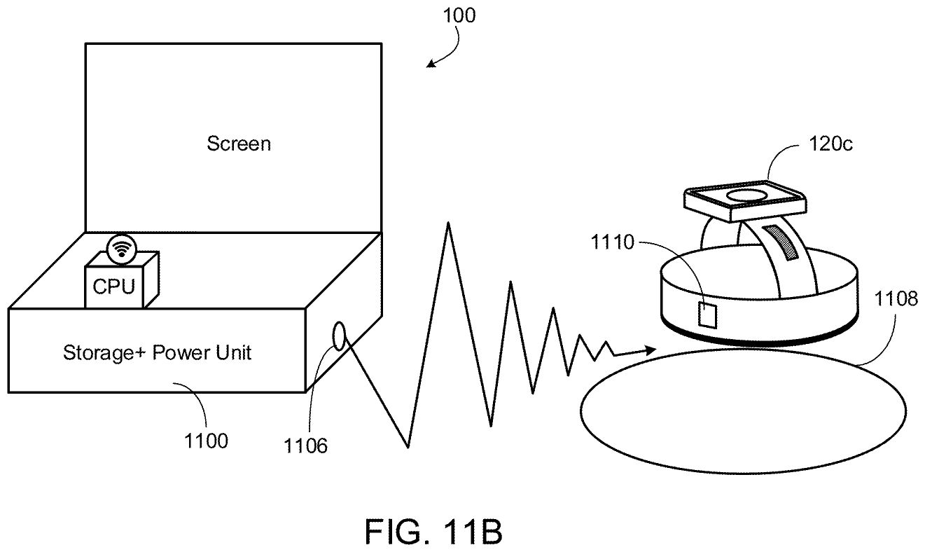

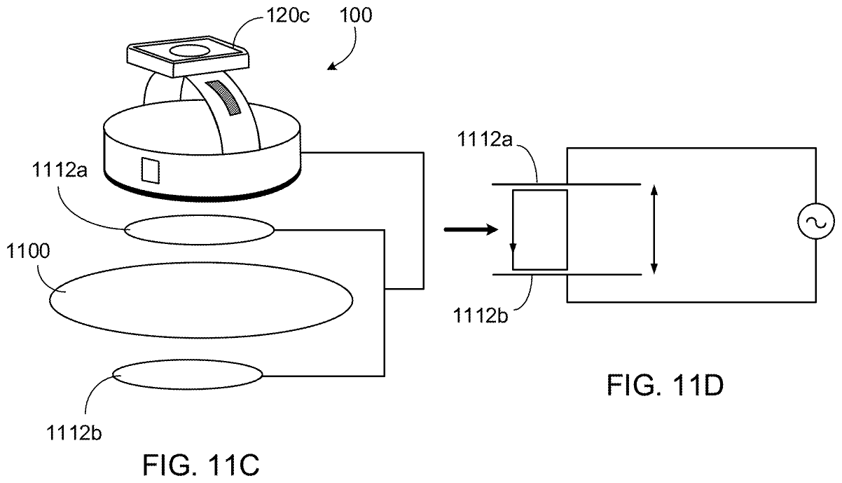

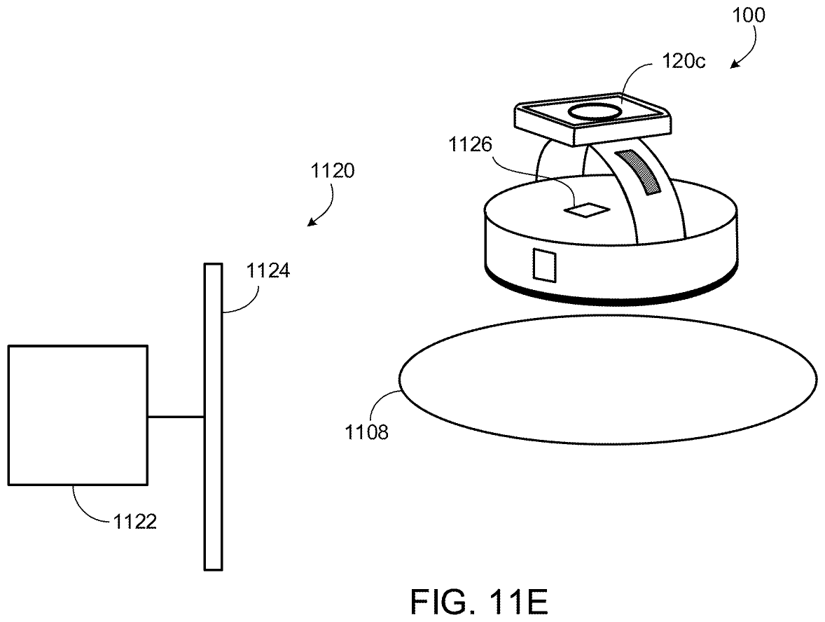

In some cases, one or more of the components of the electronic system 100 can be stored in a storage container for convenient organization and transport. For example, as shown in FIG. 11A, the components of the electronics system 100, including the computing system 110 and the wearable watches 120a-d (for simplicity, only a single watch 120a is shown) can be stored in a storage container 1100. This storage container 1100 can be placed in a convenient location, such that the electronic system 100 is readily accessible during an emergency situation. For example, the storage container 1100 can be placed in a patient treatment area, such as a patient's room or an operating room. As another example, the storage container 1100 can be placed on a moveable cart (e.g., a “crash cart”), such that the storage container 1100 can be readily relocated in the event of an emergency. In an emergency situation, members of the intervention team can access the storage container 1100, and take an appropriate wearable computing device 120a-d for use during the patient's treatment.

In some cases, the electronic system 100 can include sensors that measure the compression depth of the rescuer's chest compressions on the patient. This can be beneficial, for example, as it can provide the rescuer (as well as other members of the intervention team) with additional feedback regarding the performance of chest compressions. This information can be obtained in various ways.

For example, as shown in FIG. 11B, the storage container 1100 can include a camera 1106 positioned facing the patient's chest 1108. During the course of treatment, the rescuer places the wearable computing device 120c (mounted to the compression plate 802 through straps 804) atop the patient's chest 1108, and applies chest compressions. The compression plate 802 includes a visual marker 1110 which is detected by the camera 1106 during treatment. The visual marker 1110 can be, for example, an object or portion of the compression plate 802 of contrasting color, a light (e.g., an LED), or some other visually detectable feature. As the rescuer compresses the patient's chest, the camera 1106 records the movement of the visual marker 1110. Based on this movement, the electronic system 100 (e.g., via the computing device 110) can calculate the compression depth, and provide the rescuer with feedback regarding his performance (e.g., by transmitting the information to the wearable device 120c for presentation to the user via a network connection). In some cases, a visual reference scale (e.g., a ruler or an object of known size) can be placed near the patient and in view of the camera 1106 to calibrate the compression depth calculations.

As another example, as shown in FIG. 11C, the system 100 can include two proximity sensors 1112a-b. The first proximity sensor 1112a can be placed on the patient's chest 1108, and a second proximity sensor 1112b can be placed beneath the patient's back, opposite the patient's chest 1108. The first and second sensors can detect their proximity to one another; based on this information, the electronic system 100 (e.g., via the computing device 110) can calculate the compression depth, and provide the rescuer with feedback regarding his performance (e.g., by transmitting the information to the wearable device 120c for presentation to the user via a network connection).

In some cases, as shown schematically in FIG. 11D, the proximity sensors 1112a-b can each act as a parallel capacitor plates, and the electronic system 100 can detect their proximity to one another by detecting a change in impedance between the proximity sensors 1112a-b. Based on this change in impedance, the electronic system 100 (e.g., via the computing device 110) can calculate the compression depth, and provide the rescuer with feedback regarding his performance (e.g., by transmitting the information to the wearable device 120c for presentation to the user via a network connection).

As another example, in some cases, the system 100 can include an electromagnetic motion tracking system 1120 to measure the compression depth. As shown in FIG. 11E, electromagnetic motion tracking system 1120 includes a signal generator 1122, a transmit antenna assembly 1124 electrically coupled to the signal generator 1122, and one or more sensors 1126 embedded on or within the wearable computing device 120c (on or within the plate or pad).

During use, the signal generator 1122 applies an electrical current to the transmit antenna assembly to generate a magnetic field (e.g., a near field, low frequency magnetic field having particular known vector characteristics) about the patient's chest 1108. In turn, directional antenna assemblies embedded within each of the sensors 1126 detect the magnetic field, and transmit information regarding the detected magnetic field (e.g., the magnitude and orientation of the detected field) to a computing device (e.g., to the wearable computing device 120c or the computing device 110 through a wired or wireless connection). Based on this information, the system 1120 can determine the position and orientation of each of the sensors with respect to the transmit antenna assembly 1124. Thus, the system 1120 can monitor the changes in position of the sensors 1126 to estimate the movement of the sensors 1126 during treatment, and in turn, estimate the degree to which the patient's chest is compressed during treatment. Electromagnetic motion tracking systems can include, for example, components from Polhemus, Inc. (Colchester, Vt.), such as G4, Liberty, Patriot, Scout, Liberty Latus, Fastrak, or Patriot Wireless.

Although FIG. 11E depicts the sensors 1126 as embedded on or within the wearable computing device 120c, this is merely an illustrative example. In some cases, the sensors 1126 can be separate from the wearable computing device 120c, and can be independently positioned onto a patient's chest by a caretaker prior to treatment. For example, in some cases, the sensors 1126 can be mounted within a thin discrete housing (e.g., a mat made of a soft or compliant material), and placed between the patient's chest and the wearable computing device 120c prior to a caretaker applying chest compressions. During use, the sensors 1126 detect the magnetic field generated by the antenna assembly 1124, and information regarding the detected magnetic field to a computing device (e.g., via a wired or wireless transceiver communicatively coupled to the sensors).

Other sensors are also possible, depending on the implementation. For example, in some cases, the wearable computing device 120c can include an accelerometer. Based on changes in acceleration, the electronic system 100 (e.g., via the computing device 110) can calculate the compression depth, and provide the rescuer with feedback regarding his performance. In some cases, the compression depth can be calculated through spectral analysis of acceleration measurements obtained by the accelerometer. For example, the accelerometer can obtain a series of measurements over time (signal a(t)). This signal can be approximated as:

$$a ( t ) = {\sum }_{k = 1}^{N} {A}_{k} cos ( 2 π {kf}_{cc} t + {θ}_{k} ) .$$

where N is the number of harmonics, f_{CC} is the mean frequency of the compressions (hertz), Aₖ is the amplitude of the kth harmonic.

Correspondingly, the chest displacement can be approximated as:

$$s ( t ) = {\sum }_{k = 1}^{N} {S}_{k} cos ( 2 π {kf}_{cc} t + {φ}_{k} ) , \\ where$$

$${S}_{k} = \frac{1000 {A}_{k}}{{( 2 π {kf}_{cc} )}^{2}}$$

$$and$$

$${φ}_{k} = {θ}_{k} + π , for k = 1 , 2 , … , N .$$

To obtain signal s(t), the signal a(t) is transformed via a fast Fourier transform (FFT) to obtain signal A(t). Several harmonics (e.g., the first three harmonics) and their fundamental frequency are determined based on the transformed signal, and are used to estimate the signal s(t) based on the equations above. Accordingly, the rate and depth of the compress compressions can be calculated using the resulting signal s(t) (e.g., by determining the amplitude range of signal s(t) within one or more cycles, and determining the frequency of those cycles). Although an example technique of estimating the compression depth is described, this is merely an illustrative example. Other techniques are also possible, depending on the implementation.

As shown in FIG. 11A, the storage container 1100 also includes a power unit 1102 and a display device 1104. The power unit 1102 provides power to each of the components contained within the storage container 1100. For example, the power unit 1102 can obtain power from an external source (e.g., through a power cord 1106), convert the power as necessary, and deliver the power to the computing system 110. As another example, the power unit 1102 can deliver power to each of the wearable computing devices 120a-d and/or sensors 1002 and 1004 to recharge batteries contained within them. As another example, the power unit 1102 can deliver power to the display device 1104.

The display device 1104 visually presents information to the members of the intervention team. The types of information presented by the display device 1104 can vary, pending on the implementation. For example, in some cases, the display device 1104 can display the same or similar information as that being presented on one or more of the wearable computing devices 120a-d. This can be useful, for example, as it allows each member of the intervention team to quickly view information pertaining to any of the members of the team. As another example, in some cases, the display device 1104 can display a selection of information regarding the patient and/or the treatment procedure (e.g., the amount of time that has elapsed since the leader initiated the intervention, vital signs of the patients, the next task to be performed by a member of the team, the last task that was performed, and so forth). As another example, in some cases, the display device 1104 can display historical information (e.g., historical charts or graphs of information regarding the treatment—such as a time-dependent graph of the CPR quality index, the perfusion quality index, and/or information regarding the patient—such as a time-dependent graph of the patient's vital signs).

In some cases, the storage container 1100 can automatically engage or disengage the electronic system 100 when it is accessed. For example, in some cases, the display device 1104 can be mounted on a hinge or other articulating mechanism, such that it can open and close to reveal the wearable computing devices 120a-d stored within the storage container 1100. When the display device 1104 is swung open, the storage container 1100 can power up and/or initiate operation of the computing system 110, the wearable computing devices 120a-d, and/or the display device 1104, such that each are ready for use. This can be beneficial, for example, as it allows members of the intervention team to quickly access and operate the electronic system 100 in the event of an emergency.

In some cases, the information displayed on the wearable computing devices 120a-d and/or the display device 1104 can be color coded, such that users can more readily understand the presented information. For example, in some cases, information related to defibrillation can be color coded yellow. As another example, information related to medication administration-related events including timers, doses, and so forth can be color coded blue. As another example, information such as compression timers and alerts can be color coded red. As another example, green can be used to indicate that parameters are within an acceptable range, and to indicate information related to capnometer readings. In practice, other combinations of colors and information can be used, depending on the implementation.

Although implementations of electronic system 100 are shown often, these are merely illustrative examples. In practice, the electronic system 100 can differ, depending on the implementation.

For example, although the electronic system 100 is described as having four wearable electronic devices 120a-d, the number of wearable electronic devices can vary. For example, in some cases, there may be additional roles in addition to or instead of those described. Thus, there may be one or more wearable electronic devices corresponding to each of those roles. Similarly, in some cases, more than one wearable electronic devices may correspond to some or all of the roles. This can be beneficial, for example, if multiple people are assigned to the same role as a team.

Further, not all wearable electronic devices 120a-d need be used simultaneously. For instance, in some cases, one or more of the roles may be left unfilled, and the responsibilities and tasks associated with those roles can be assigned to other members of the intervention team and/or automatically performed by the electronic system 100. As an example, in some cases, an intervention team might not have a recorder. Thus, the tasks associated with the recorder can be assigned to other members of the team (e.g., the leader) and/or automatically performed (e.g., by automatically recording the occurrence of each event without user intervention). In this manner, the electronic system 100 can provide members of the team with information regarding their tasks, regardless of the number and members available to assist in a given situation.

Further, in some cases, the computing device 110 need not be present. Instead, the functionality of the computing device 110 can instead be performed by one or more of the wearable computing devices 120a-d. For example, in some cases, the wearable computing device 120a receives information from each of the other wearable computing devices 120b-d, and stores the received information for future retrieval. Thus, in these cases, the wearable computing device 120a can act as a central location for the storage of information collected by the system 100.

Further, in some cases, the wearable computing device 120c can be used independently from the other devices of the system 100. For example, the wearable computing device 120c can provide a user with instructions for treating a patient in cardiac arrest without the assistance of others, provide feedback to the user during the course of treatment, call others for assistance, send a command to the chest compression device 131 to begin performing chest compressions on the patient, and send a command to the defibrillator 132 to begin administering defibrillating shocks to the patient. This can be beneficial, for example, in situations where a full medical intervention team is not immediately available to assist the patient. This can also be useful, for example, in a home, office, or public setting (e.g., a restaurant, theater, store, or so forth), where those who are immediately available to assist the patient may be lay people, and may lack to experience needed to treat the patient without guidance.

In these cases, the wearable computing device 120c can perform some or all of the functions that might otherwise be performed by other components of the system 100. For example, the wearable computing device 120c can retrieve information regarding one or more treatment protocols suitable for a single person to perform. The wearable computing device 120c can provide the user with information regarding when to perform each of the tasks to the protocol, the proper time at which to perform those steps.

Further, the wearable computing device 120c can provide instructions to assist the user in performing the tasks. For instance, the wearable computing device 120c can present the user with text, images, videos, animations, spoken information, and/or audio cues that guide the user in performing each task. As examples, the wearable computing device 120c can present the user with text and audio prompting the user to take the patient's pulse, present the user with and text and animation regarding the proper technique for doing so. The wearable computing device 120c can also present spoken instructions that guide the user through each steps of the technique. Further, the wearable computing device 120c can prompt the user for input regarding the outcome of the technique, such that it can determine which task should be performed next. In this manner, the user is guided through the entire treatment process.

In some cases, the wearable computing device 120c can also provide the user with feedback regarding his performance, and if needed, instruct the user in adjusting his efforts. For example, the wearable computing device 120c can determine whether the user is applying a proper amount of pressure during chest compressions, and whether the user is applying chest compressions at the proper rate. If the user is not performing in accordance with the protocol, the wearable computing device 120c can instruct the user to adjust his performance, and provide instructions for making those adjustments.

In some cases, the wearable computing device 120c can also record the performance of the user during the treatment process. For example, the wearable computing device 120c can record which tasks were performed by the user, the time at which those tasks were performed, and information regarding the performance of those tasks (e.g., the amount of pressure applied, the rate at which chest compressions were applied, and so forth). This information can be accessed later to assess the user's performance and/or to provide caretakers with additional information that could be useful in treating the patient in the future.

In some cases, the wearable computing device 120c can also call others for assistance. For example, the wearable computing device 120c can transmit a message to an emergency call center, hospital, a fire station, and/or a police station, notifying the recipients that an emergency situation is in progress. The message can be, for example, a text message (e.g., SMS message), an e-mail, an instant message, a telephone call, a facsimile, or other message. In some cases, the wearable computing device 120c can also transmit its location (e.g., by obtaining location information using a GPS sensor). This can be useful, for example, as it frees the user to immediately treat the patient without delay. Additionally it allows the rescuer to perform resuscitation tasks without the need to hold a telephone and can therefore receive instructions from a dispatcher without interrupting compressive efforts. This can also be useful, for example, as it automatically notifies others of the situation, without relying on a user who might otherwise forget or delay initiating the notification process to others.

In some cases, the wearable computing device 120c can also send a command to the chest compression device 131 to begin performing chest compressions on the patient and/or send a command to the defibrillator 132 to begin administering defibrillating shocks to the patient. This can also be useful, for example, as it automatically begins treating the patient, without relying on a user's skill in manually treating the patient.

Furthermore, although computing devices 120a-d are described as wearable, in some implementations, the devices 120a-d may include smart phones, tablets or other portable or handheld smart computing devices that are not necessarily wearable by users. In some implementations, the computing devices 120a-d are wearable and are also supplemented with an additional portable computing device, such as a smart phone or smart tablet. For instance, in some cases, data recorded by the wearable device, such as compression depth and heart rate, may be transmitted (e.g., via Bluetooth communication) to the supplemental computing device for presentation of the data on a larger display. Alternatively, or in addition, the supplemental computing device may include specific applications for manipulating and analyzing the data transmitted from the wearable computing device. For instance, the supplemental computing device may include an application that has access to detailed medical history for the patient information or an application that can generate a detailed illustrated report based on the condition of the patient over the course of treating the patient. In some implementations, the wearable computing devices 120a-d include an application that can generate the detailed illustrated report based on the condition of the patient, as well as the patient history. The report generated by either the supplemental computing device or the wearable computing device may pull patient data from an electronic medical record, time-stamped data from one or more of the wearable computing devices and/or from one or more sensors coupled to the patient, as well as the real time data capture from the one or more wearable computing devices. The compiled report may be transmitted back to the electronic medical record or be used for training purposes.

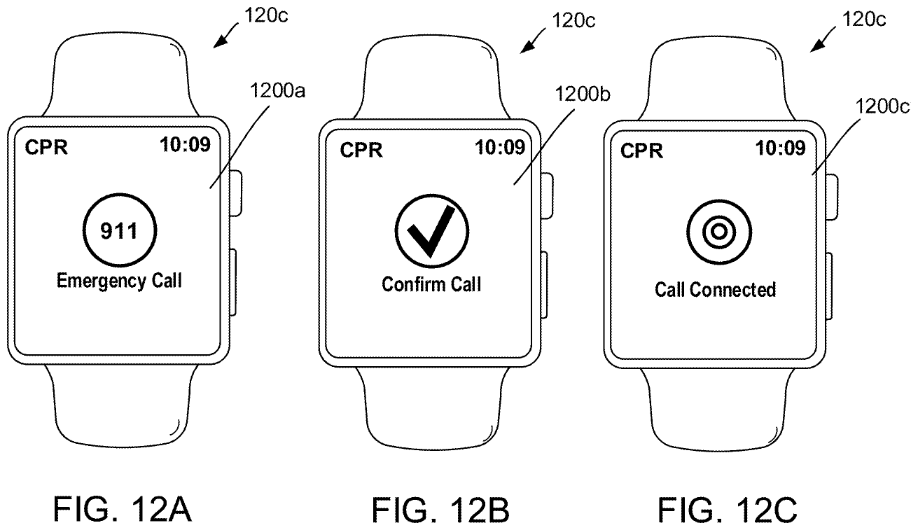

FIGS. 12A-C show example GUIs 1200a-c that can be displayed to the user during the notification process. For example, as shown in FIG. 12A, the wearable computing device 120c can present a GUI 1200a indicating that an emergency call should be placed. As shown in FIG. 12B, the wearable computing device 120c can present a GUI 1200b confirming to the user that the emergency call is being placed. As shown in FIG. 12C, the wearable computing device 120c can present a GUI 1200c indicating that that the call is connected. In this manner, the wearable computing device 120a-c can inform the user of each step of an emergency, and can keep the user apprised of the call's status.

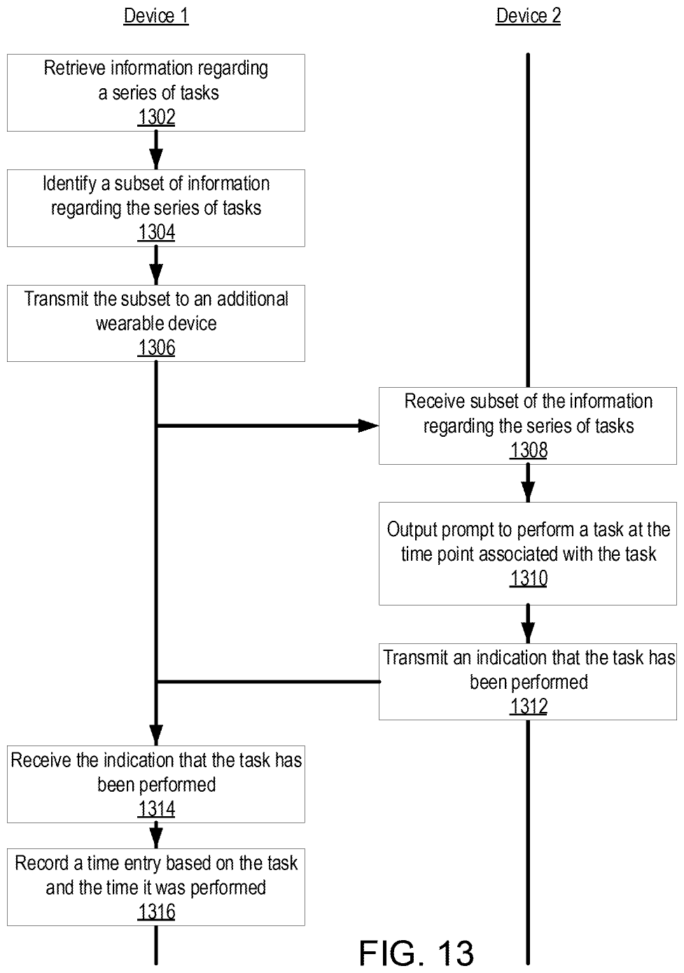

FIG. 13 is a flowchart of an example process 1300 for assisting one or more users in treating a patient in cardiopulmonary arrest. In this simplified example, the steps performed by two wearable computing devices are described. However, it is understood that the steps can be performed by more than two wearable computing device (e.g., three, four, or more). The process 1300 begins by a first wearable computing device (e.g., wearable computing device 120a) retrieving information regarding a series of tasks to be performed in treating a patient in cardiopulmonary arrest (step 1302). This information can include, for example, information regarding one or more intervention protocols that an intervention team can follow to treat the patient. Information regarding each protocol can be retrieved, for example, from an external computer system (e.g., a computing system 110), and/or pre-stored on the first wearable computing device. Each tasks can be a task that is performed to treat the patient in cardiopulmonary arrest. Example tasks include measurement of a pulse of the patient, administration of a drug to the patient, performance of chest compressions on the patient, and performance of a defibrillating shock to the patient.

The information regarding the series of tasks can include information regarding each of the tasks. For example, the information can include, for each task, a description of the task, instructions for performing the task, an indication of a user that is assigned to perform the task, and/or an indication to perform the task at a particular time.

The first wearable computing device then identifies a subset of the information regarding the series of tasks (step 1304), and transmit the subset of information to a second wearable computing device (e.g., a wearable computing device 120b-c) (step 1306). In some cases, the subset of information can pertain to a particular task assigned to the wearer of the second wearable computing device. For example, the subset of information can include a description of the task to be assigned to the wearer of the second wearable computing device, instructions for performing that task, and/or an indication to perform the task at a particular time. The subset of information can be transmitted over a suitable communications network (e.g., a Wi-Fi network, a Bluetooth network, a NFC network, and so forth).

In response, the second wearable computing device receives the subset of the information regarding the series of tasks (step 1308), and outputs a prompt to perform a task at the time point associated it the task (step 1310). The task can be, for example, the task assigned to the user of the second wearable computing device. The prompt can include, for example, a visual indication (e.g., an image, text, an animation, a “pop-up,” a change in color, or other visual indication), an audible indication (e.g., a sound effect, music, or other audible indication), and/or a haptic indication (e.g., a vibration). The prompt can also include, for example, a description of the task to be performed and/or instructions to performing the task.

The second wearable computing then transmits an indication that the task has been performed (step 1312). The indication can include, for example, a name of description of the task performed, a time at which the task was performed, and/or further information regarding the task (e.g., one or more performance parameters associated with the task). In some cases, the second wearable computing device can determine that the task has been performed based an input from a user (e.g., a wearer of the first wearable computing device, the wearer of a second wearable computing device, or other user). The indication can be transmitted over a suitable communications network.

In response, the first wearable computing device receives the indication (step 1314), and records a time entry based on the task and the time it was performed (step 1316). For example, the first wearable computing device can record a name of description of the task performed, a time at which the task was performed, and/or further information regarding the task in a storage device located on the first wearable computing device, or on a storage device located external to the first wearable computing device (e.g., a computing device 110).

Although process 1300 describes the steps performed by two wearable computing devices, this is a merely a simplified example to demonstrate the concept. In practice, these steps can be performed by more than two wearable computing devices. For example, the process 1300 can be performed by four wearable computing devices. The first wearable computing device (e.g., the wearable computing device 120a) can retrieve information regarding a series of tasks. The information can include, for each task, an indication of a particular user from among several users to perform the task, an indication of a time point for that particular user to perform the task.

Based on this information, the first wearable computing device can identify multiple subsets of information regarding the tasks, each subset of information corresponding to a different one of the tasks. The first wearable computing device can identify which steps are assigned to which wearers of the other wearable computing devices (e.g., wearable computing devices 120b-d), and transmit each subset of information to the appropriate wearable computing device.

In response, each of the additional wearable computing devices receives the respective subset of information, and outputs a prompt to its wearer perform a task at the time point associated it the task. Each additional wearable computing device then transmits an indication that the task has been performed to the first wearable computing device. In response, the first wearable computing device receives the indications, and record time entries based on the tasks and the time they were performed.

### Examplary Application

In an exemplary application, upon arriving at a scene where a patient in need of cardiopulmonary resuscitation is needed, the rescue team member or members chosen by the team leader to perform compressions will place the chest compression device (e.g., any of the chest compression devices as described herein) on the patient's chest. Team members may also connect one or more patient sensors as described herein to the chest compression device. For example, referring to FIG. 15, one or more sensors may be be placed in order on the patients body as follows: a sensor 1502 on the forehead for brain oxygenation; a sensor 1504 attached to the respiratory bag-mask assembly to measure end tidal CO₂ and respiration rate; a sensor 1506 applied to the patient's chest to obtain an ECG signal; and a sensor 1508 applied to the patient's body to assess perfusion. These sensors will serve as biofeedback to assess the resuscitative efforts and are communicatively coupled to the chest compression device 1500 either wirelessly or through wires connections. The chest compression device 1500 may include one of the wearable computing devices 120a-120d releasably secured to the device 1500. In a manual mode, a team member will place their hands in CPR interlocking position and press firmly and rhythmically on the chest compression device 1500. As described herein, the screen of the wearable computing device attached to the chest compression device 1500 may face the team member or members directly and can display information relevant to the compression such as compression rate, pressure applied, and a compression quality index. The team member or members concentrate on the rate and pressure applied to the chest to keep the quality index in the appropriate zone. In some implementations, the wearable computing device attached to the chest compression device 1500 may output to its display a warning. For example, in some cases, it will issue a warning periodically (e.g., every 2 minutes) to suggest switching the team member that is applying the compression.

Alternatively, a team member may configure the chest compression device 1500 to operate in automatic mode. For example, a team member may attach a compression bladder, as described herein, to the bottom of the chest compression device using a hook and loop fastener. The team member may connect the compression bladder to a air compressor that also is electronically coupled to the chest compression device 1500. Under automatic operation, a team member may issue a command on their wearable computing device 120a to begin compressions and set a desired compression rate and pressure. A command then may be issued wirelessly to the chest compression device 1500 from the wearable computing device 120a. Upon receiving the command, the chest compression device 1500 will operate the air compressor so that compressions are delivered to the patient at a desired rate and chest displacement (e.g., 120 times a minute and a chest displacement of 2 inches in accordance to ACLS guidelines). The chest compression device 1500 may monitor the chest displacement from a displacement sensor and adjust the compression pressure as necessary to achieve the desired chest displacement. Alternatively, information about chest displacement can be transmitted from the displacement sensor to the chest compression device and then to one of the wearable computing devices 120a-120d, where the team leader can view the chest displacement and, if necessary, modify the desired pressure to be applied. In some cases, modifications to rate and depth may be performed by the operator also to achieve the desired perfusion or other desired parameter.

In some implementations, the ECG sensor 1506 may detect a shockable rhythm. In such cases, the measurement of the shockable rhythm will be transmitted to the chest compression device 1500, and in turn, to one of the wearable computing devices 120a-120d. In some cases, if the shockable rhythm is within a predetermined range, the wearable computing device 120a-120d may output a recommendation to the team leader to apply a shock to the patient. The team leader may, in response, choose the desired shock settings such as synchronized/non-synchronized, and the desired joules. Upon selecting the desired settings, the team leader may issue a command through the wearable computing device 120a-120d to apply a shock to the patient. Upon receiving the command, the wearable computing device may transmit the command to the chest compression device, which, in turn, may cause the defibrillator to apply the shock having the selected parameters to the patient.

In some implementations, one or more of the wearable computing devices 120a-120d may output to their respective displays a message and/or warning prior to the shock being applied. For example, after 2 minutes, one or more of the devices 120a-120d may output a warning to alert the person doing compressions that a shock will be administered. Once the shock is complete, one or more of the devices 120a-120d may output to their respective display a message to check for a pulse and resume compressions.

Within the integrated system the chest compression device 1500 can share compression data with the wearable computing device operated by the team leader. The sensor data collecting biometric information regarding the patient's perfusion also may be transmitted to the wearable computing device operated by the team leader. The team leader can then choose to instruct the rescuer performing chest compressions to modify or continue with compressions as being performed.

Some implementations of subject matter and operations described in this specification can be implemented in digital electronic circuitry, or in computer software, firmware, or hardware, including the structures disclosed in this specification and their structural equivalents, or in combinations of one or more of them. For example, in some implementations, computing device 110, wearable computing devices 120a-d, chest compression device, and defibrillator 132 can be implemented, in part, using digital electronic circuitry, or in computer software, firmware, or hardware, or in combinations of one or more of them. In another example, process 1300 can be implemented using digital electronic circuitry, or in computer software, firmware, or hardware, or in combinations of one or more of them.

Some implementations described in this specification can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

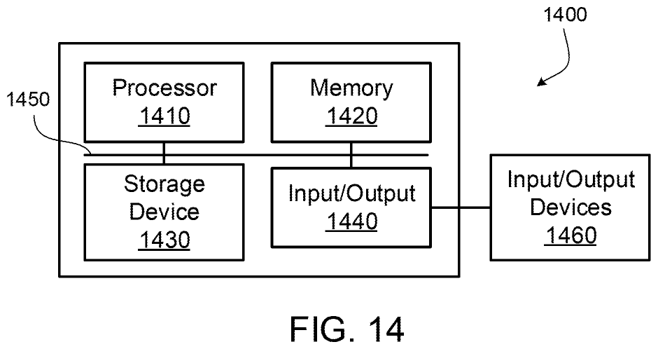

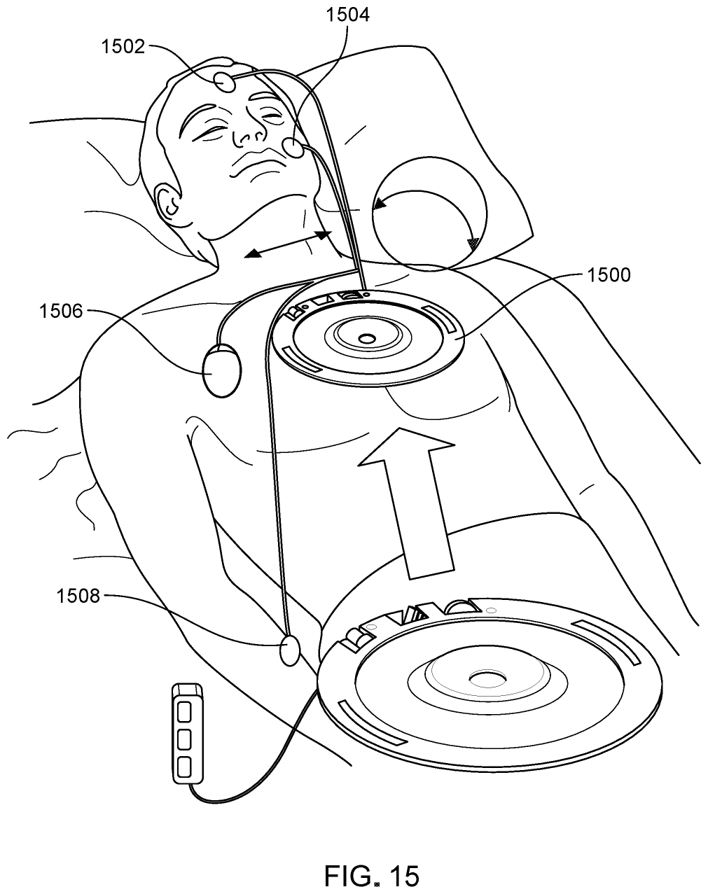

FIG. 14 shows an example computer system 1400 that includes a processor 1410, a memory 1420, a storage device 1430 and an input/output device 1440. Each of the components 1410, 1420, 1430 and 1440 can be interconnected, for example, by a system bus 1450. The processor 1410 is capable of processing instructions for execution within the system 1400. In some implementations, the processor 1410 is a single-threaded processor, a multi-threaded processor, or another type of processor. The processor 1410 is capable of processing instructions stored in the memory 1420 or on the storage device 1430. The memory 1420 and the storage device 1430 can store information within the system 1400.

The input/output device 1440 provides input/output operations for the system 1400. In some implementations, the input/output device 1440 can include one or more of a network interface devices, e.g., an Ethernet card, a serial communication device, e.g., an RS-232 port, and/or a wireless interface device, e.g., an 802.11 card, a 3G wireless modem, a 4G wireless modem, etc. In some implementations, the input/output device can include driver devices configured to receive input data and send output data to other input/output devices, e.g., keyboard, printer and display devices 1460. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable subcombination.

A number of implementations have been described. Nevertheless, it will be understood that various modifications may be made without departing from the spirit and scope of the invention. Accordingly, other implementations are within the scope of the following claims.

What is claimed is:

1. A system comprising:
a first computing device; and
a chest compression device configured to communicate with the first computing device,
wherein the chest compression device comprises a defibrillator, and
wherein the first computing device is configured to obtain information regarding a patient being treated for cardiopulmonary arrest and to send commands to the chest compression device, wherein the commands comprise a defibrillator activation command to activate the defibrillator.

2. The system of claim 1, wherein the chest compression device comprises:
a compression plate;
a compression bladder; and
at least one adjustable strap coupled to the compression plate and for securing the chest compression device to the patient.

3. The system of claim 2, wherein the defibrillator comprises:
a first defibrillator pad attached to the compression bladder; and
a second defibrillator pad attached to the at least one adjustable strap.

4. The system of claim 3, wherein the compression bladder is configured to be removably secured to the compression plate.

5. The system of claim 4, wherein the compression bladder is secured to the compression plate through a hook and loop fastener.

6. The system of claim 2, wherein the chest compression device comprises a gel pack.

7. The system of claim 2, wherein an internal space of the compression bladder comprises a gel or liquid.

8. The system of claim 2, further comprising a compressor couplable to the compression bladder, wherein during operation of the system, the compressor is operable to inflate and deflate the compression bladder.

9. The system of claim 1, wherein the chest compression device comprises a power source coupled to a first defibrillator pad.

10. The system of claim 9, wherein the chest compression device is configured to cause the power source to apply a voltage potential to the first defibrillator plate upon receiving the defibrillator activation command from the first computing device.

11. The system of claim 1, wherein the chest compression device comprises an accelerometer configured to measure an acceleration of the chest compression device during operation of the chest compression device, and
wherein the first computing device is configured to obtain, from the accelerometer, an acceleration signal indicative of the acceleration and to derive a compression depth associated with treating the patient based on the acceleration signal.

12. The system of claim 11, wherein the accelerometer is embedded within the compression plate.

13. The system of claim 11, wherein the chest compression device is operable to automatically apply pressure to the patient periodically.

14. The system of claim 1, further comprising a capnometer operable to sense a concentration of carbon dioxide exhaled by the patient, wherein the capnometer is couplable to the first computing device.

15. The system of claim 1, further comprising an oxygen sensor operable to sense an amount of oxygen perfusion of the patient, wherein the oxygen sensor is couplable to the first computing device.

16. The system of claim 1, further comprising a tissue perfusion sensor operable to sense an amount of tissue perfusion of the patient, wherein the tissue perfusion sensor is couplable to the first computing device.

17. The system of claim 1, further comprising a brain oxygenation sensor operable to sense an amount of brain oxygenation of the patient, wherein the brain oxygenation sensor is couplable to the first computing device.

18. The system of claim 1, further comprising a blood pressure sensor operable to sense a blood pressure of the patient, wherein the blood pressure sensor is couplable to the first computing device.

19. The system of claim 1, wherein the chest compression device comprises an angular rate sensor.

20. The system of claim 1, wherein the chest compression device comprises one or more light emitting elements.

21. The system of claim 1, wherein the chest compression device comprises:
an upper compression plate;
a lower compression plate;
a mechanical piston between the upper compression plate and the lower compression plate and configured to increase a distance between the upper compression plate and the lower compression plate during operation of the chest compression device.

22. The system of claim 1, further comprising:
a strap coupled to the chest compression device; and
a strap tensioner for receiving a first end of the strap and a second end of the strap such that the strap is arranged in a loop, wherein, during operation of the the chest compression device, the strap tensioner is operable to increase or decrease a length of the loop.

23. A system comprising:
a first computing device; and
a chest compression device configured to communicate with the first computing device,
wherein the chest compression device comprises a defibrillator,
wherein the first computing device is configured to obtain information regarding a patient being treated for cardiopulmonary arrest and to send commands to the chest compression device, wherein the commands comprise a defibrillator activation command to activate the defibrillator, and
wherein the first computing device is further configured to determine, from the patient information, a patient state indicative of cardiac dysrhythmia, wherein the first computing device, responsive to determining the patient state indicative of cardiac dysrhythmia, outputs to a display of the first computing device a recommendation to activate or deactivate the defibrillator.

24. A method comprising:
receiving, at a first computing device in a chest compression device comprising a defibrillator, a patient attribute signal measured by a patient sensor during operation of the chest compression device;
determining, from the patient attribute signal, a patient state;
responsive to determining the patient state, outputting to a display of the first computing device, a user prompt;
receiving, as an input to the first computing device, a user command to activate the defibrillator; and
responsive to receiving the user command to activate the defibrillator, transmitting a defibrillator activation command to the defibrillator.

25. The method of claim 24, wherein the patient attribute signal indicates a blood pressure of the patient.

26. A method comprising:
receiving at a first computing device, from a chest compression device comprising (a) a defibrillator and (b) an accelerometer configured to measure an acceleration of the chest compression device during operation of the chest compression device, the acceleration measured by the accelerometer;
determining, from the accelerometer, a compression depth; and
responsive to determining the compression depth, outputting to a display coupled to the first computing device a recommendation an indication of a compression quality.

27. The method of claim 26, further comprising:
receiving, at the first computing device and from a sensor, patient heart rate information;
determining that the heart rate information is below a predetermined acceptable range of heart rates; and
responsive to determining that the heart rate information is below a predetermined acceptable range of heart rates, activating the defibrillator.

28. The method of claim 27, further comprising outputting, prior to activating the defibrillator, a warning that a defibrillation shock is about to be applied.
